SEQUENCE LISTING (2 sequences)

SEQ ID NO: 1 - DNA, 3512 bp, Homo sapiens
gggtgtggcctccgggcggcatggctgcttctcaggtgatgccggcttcagctagtggggtctagttgaccgttccgcagccgccagggccagcggaaagccggtcagggggaaccgcggcggggctggtgtcatgagcctgaggtgaacttgagggtgcctcctcagcggtctcccgccctgccctgaggggcgccgggaccccaaagagcggaggaagagcgccaccccgacggccaccgcttcggagccagcacgcggggtaccctacggggagcgcggatgcccccgtgttcgggcggggacggctccacccctcctgggccctcccttcgggacagggactgtcccgcccagagtgctgaatacccgcgcgaccgtctggatccccgcccaggaagcccctctgaagcctcctcgccgccgtttctgagaagcagggcacctgttaactggtaccaagaaaaggcccaagtgtttctctggcatctgttggtgtctggatccaccactctactctgtctctggaaacagcccttccacgtctctgcattccctgtcactgcgtcactggccttcagacagagccaaggtgcagggcaacacctctacaaggatctgcagccatttatattgcttaggctactgatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaactcatcagatgcattggacaaaatccggtatgaaagcttgacagatcccagtaaattagactctgggagagagctgcatattaaccttataccgaacaaacaaggtcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaatatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggtttattttcacagattggggtaacgtgcactgtaagacgtatgtaacatgatgttaactttgtgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaaaataactcagacccagtcttgtggatggaaatgtagtgctcgagtcacattctgcttaaagttgtaacaaatacagatgagttaaaag

SEQ ID NO: 2 - protein, 854 aa, Homo sapiens
MPPCSGGDGSTPPGPSLRDRDCPAQSAEYPRDRLDPRPGSPSEASSPPFLRSRAPVNWYQEKAQVFLWHLLVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGRELHINLIPNKQGRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

(19) United States
(12) Patent Application Publication
(10) Pub. No.: US 2012/0165403 A1
(43) Pub. Date: Jun. 28, 2012

(54) METHODS OF INHIBITING THE ACTIVITY OF HSP90 AND/OR ARYL HYDROCARBON RECEPTOR

(75) Inventors: Thomas A. Gasiewicz, Victor, NY (US); Christine Palermo, Rochester, NY (US)

(73) Assignee: UNIVERSITY OF ROCHESTER, Rochester, NY (US)

(21) Appl. No.: 13/316,092

(22) Filed: Dec. 9, 2011

Related U.S. Application Data
(63) Continuation of application No. 11/718,674, filed on Aug. 1, 2007, now abandoned.
(60) Provisional application No. 60/625,515, filed on Nov. 5, 2004.

(51) Int. Cl. A61K 31/353; C12N 5/09; A61P 35/00

(52) U.S. Cl. 514/456; 435/375

(57) ABSTRACT

The present invention relates to a method of screening compounds for binding to hsp90 by exposing a compound to hsp90 or a polypeptide fragment thereof containing amino acid residues 538-728 of the full length protein and determining whether the compound binds to hsp90 of the polypeptide fragment thereof. Also disclosed is a method of screening compounds for inhibition of hsp90 activity. The present invention further relates to a method of screening compounds as a cancer therapeutic and a method of treating cancerous conditions. Also disclosed is a method of inhibiting transcription-inducing activity of an aryl hydrocarbon receptor in a cell and a method of modifying expression of a gene that is activated by an aryl hydrocarbon receptor.

This application is a continuation of U.S. patent application Ser. No. 11/718,674, filed Aug. 1, 2007, which is a national stage application under 35 U.S.C. §371 from PCT Application No. PCT/US2005/040114, filed Nov. 7, 2005, which claims the benefit of U.S. Provisional Patent Application Ser. No. 60/625,515, filed Nov. 5, 2004, which are hereby incorporated by reference in their entirety.

This invention was made with government support under grant numbers ES09702, ES07026, and ES01247 awarded by the NIH. The government has certain rights in this invention.

### FIELD OF THE INVENTION

This invention relates to methods of screening compounds, methods of preventing or treating cancer in a subject, as well as methods of inhibiting the activity of heat shock protein 90 and aryl hydrocarbon receptor transcription in a cell.

### BACKGROUND OF THE INVENTION

The Aryl Hydrocarbon Receptor (“AhR”) is a ligand-dependent transcription factor that can be activated by numerous structurally diverse synthetic and naturally occurring compounds such as polycyclic aromatic hydrocarbons, indoles, and flavonoids. In an unliganded state, the AhR is present in a latent conformation in the cytoplasmic compartment of the cell associated with two molecules of the molecular chaperone heat shock protein 90 (“hsp90”) (Perdew, J. Biol. Chem. 263:13802-13805 (1988) and Wilhelmsson et al., EMBO J. 9:69-76 (1990)), an immunophilin-like protein, XAP2 (Carver et al., J. Biol. Chem. 272:11452-11456 (1997); Ma et al., J. Biol. Chem. 272:8878-8884 (1997); and Meyer et al., Mol. Cell. Biol. 18:978-988 (1998)), and the hsp90 interacting protein, p23 (Kazlauskas et al., J. Biol. Chem. 274:13519-13524 (1999)). Ligand binding initiates a cascade of poorly characterized events involving translocation to the nucleus, release of hsp90, and heterodimerization with ARNT (Schmidt et al., Annu. Rev. Cell. Dev. Biol. 12:55-89 (1996) and Rowlands et al., Crit. Rev. Toxicol. 27:109-134 (1997)). The ligand bound AhR-ARNT complex is capable of recognizing consensus sequences termed dioxin-response elements (“DRE”s) located in the promoter region of CYP1A1 and other responsive genes, thereby activating transcription (Schmidt et al., Annu. Rev. Cell. Dev. Biol. 12:55-89 (1996) and Rowlands et al., Crit. Rev. Toxicol. 27:109-134 (1997)).

Hsp90 has been shown to be an essential component of the AhR signaling pathway. Its presence has been demonstrated to be necessary in both the proper folding and stability of the AhR complex (Carver et al., J. Biol. Chem. 269:30109-30112 (1994) and Whitelaw et al., Proc. Natl. Acad. Sci. 92:4437-4441 (1995)). Additionally, the hsp90-AhR interaction represses AhR activation either through potential steric interference with ARNT dimerization (Coumailleau et al., J. Biol. Chem. 270:25291-25300 (1995) and Perdew et al., Biochem. Mol. Int. 39:589-593 (1996)), or by interfering with the interaction between the C-terminal transactivation domains or other putative cofactors (Whitelaw et al., Mol. Cell. Biol. 14:8343-8355 (1994)). However, it remains unclear what role this protein serves in nuclear translocation. For example, the detection of an hsp90-AhR complex in the nucleus of 2,3,7,8-tetrachlorodibenzo-p-dioxin (“TCDD”) exposed cells (Wilhelmsson et al., EMBO J. 9:69-76 (1990) and Perdew, Arch. Biochem. Biophys. 291:284-290 (1991)) strongly implies that hsp90 dissociation may not be required for nuclear import. Conversely, deletion of the PAS domain of the AhR has been shown to result in ligand-independent nuclear translocation of the AhR (Ikuta et al., J. Biol. Chem. 273:2895-2904 (1998)), suggesting the association of hsp90 with the PAS domain prevents the unliganded AhR from entering the nucleus. Based on this and other data, it remains unclear whether dissociation of hsp90 is necessary for nuclear import of the AhR or whether its dissociation regulates dimerization with ARNT within the nuclear compartment of the cell. There also remains ambiguity concerning how and when the many other identified AhR-associated proteins, such as p23, XAP2, p60, hsp70, and p48, affect the AhR signaling pathway.

One approach to understanding events required for AhR activation is by delineating mechanisms involved in turning this signaling pathway off. Currently, very little is known regarding the mechanism of action of AhR antagonists. Two of the most potent and well-characterized AhR antagonists include the synthetic flavonoid, 3′-methoxy-4′ nitroflavone (“3M4NF”), and the indole derivative 3,3′-diindolylmethane (“DIM”). These compounds have been shown to function through direct competition for binding to the AhR ligand binding site (Henry et al., Mol. Pharmacol. 55:716-725 (1999); Hestermann et al., Mol. Cell. Biol 23:7920-7925 (2003)). Interestingly, the fate of the AhR upon binding of these structurally distinct antagonists is very different. Binding of 3M4NF to the AhR inhibits TCDD-mediated nuclear localization, ARNT dimerization, and DNA binding (Henry et al., Mol. Pharmacol. 55:716-725 (1999)). 3M4NF is believed to inhibit a conformational change within the AhR complex necessary for exposure of the nuclear localization sequence, resulting in retention of the AhR in the cytoplasmic compartment of the cell. Conversely, binding of DIM to the AhR allows nuclear localization, ARNT dimerization, and subsequent DNA binding. However, unlike the TCDD-bound AhR-ARNT dimer, this DIM-bound complex is incapable of recruiting the necessary co-factors responsible for initiating transcription (Hestermann et al., Mol. Cell. Biol 23:7920-7925 (2003)). These findings strongly support the hypothesis that antagonists affect AhR conformation differently than agonists, and provide evidence that structurally diverse antagonists are capable of altering the activation process very differently.

Based on the above observations and what is known about the AhR signal transduction pathway, it is conceivable that an antagonist could interfere with the AhR at numerous stages. These include: 1) prevention of release of associated proteins such as hsp90 from the complex; 2) prevention of the association of the ligand-bound AhR with ARNT; and 3) formation of a complex which includes ARNT, but lacks DRE binding ability. In addition, a compound could potentially antagonize AhR activation through indirect processes that do not involve direct binding to the AhR (i.e., ligand independent) including: 1) direct inhibition of the proteins involved in nuclear import; 2) direct binding to an associated AhR chaperone protein; 3) inhibition of kinases involved in phosphorylation events; and 4) increasing protein degradation.

Previous studies have implicated the green tea (“GT”) compound epigallocatechin gallate (“EGCG”) to have AhR antagonist activity (Palermo et al., Chem. Res. Toxicol. 16:865-872 (2003); Williams et al., Chem-Biol. Interact. 128:211-229 (2000); and Fukuda et al., J. Agric. Food Chem. 52:2499-2509 (2004)). The goal of these studies is to elucidate the molecular mechanism and consequence of this inhibition. If EGCG were functioning as a competitive antagonist, it would be important to determine how this was altering the AhR-protein complex. Conversely, if EGCG were functioning through a ligand-independent mechanism, it would be important to identify the protein target. Based on the structural similarity between EGCG and the known AhR antagonist 3M4NF, it would be expected that EGCG functions through a similar mechanism involving competition for binding to the AhR ligand binding site. Surprisingly, this is not the case.

The present invention is directed to overcoming these and other deficiencies in the art.

### SUMMARY OF THE INVENTION

A first aspect of the present invention relates to a method of screening compounds for binding to hsp90 that includes the steps of: exposing a compound to hsp90 or a polypeptide fragment thereof comprising an ATP-binding site, such as a fragment comprising amino acid residues 538-728 of the full length hsp90; and determining whether the compound binds to hsp90 or the polypeptide fragment thereof.

A second aspect of the present relates to a method of screening compounds for inhibition of hsp90 activity that includes the steps of: contacting a cell with a compound that induces AhR-regulated gene expression and a test compound that binds to hsp90 (or has otherwise been identified by the method according to the first aspect of the present invention); and then determining whether, in the presence of hsp90, said contacting is effective to inhibit AhR-induced transcription of a gene containing a dioxin response element, wherein inhibition of AhR-induced expression of the gene indicates the compound can inhibit hsp90 activity required for AhR-induced transcription.

A third aspect of the present invention relates to a method of screening compounds as a cancer therapeutic by performing the method according to the second aspect of the present invention, wherein inhibition of AhR-induced expression of the gene further indicates the compound is a potential cancer therapeutic. Compounds screened in this manner can then be tested via in vitro cell-based assays and/or in vivo animal studies for efficacy as a cancer therapeutic.

A fourth aspect of the present invention relates to a method of treating a cancerous condition that includes the step of inhibiting an interaction between hsp90 and a protein that is a causative agent of a cancerous condition, whereby said inhibiting modifies the activity of the protein that is a causative agent of the cancerous condition and thereby treats the cancerous condition.

A fifth aspect of the present invention relates to a method of inhibiting transcription-inducing activity of an aryl hydrocarbon receptor in a cell, said method including the step of contacting a cell with a polyphenol under conditions effective to bind hsp90 and form an hsp90-polyphenol complex, wherein the complex binds to the aryl hydrocarbon receptor and inhibits transcription-inducing activity of the aryl hydrocarbon receptor in the cell.

A sixth aspect of the present invention relates to a method of modifying expression of a gene that is activated by an aryl hydrocarbon receptor, said method including the step of contacting a cell with a polyphenol under conditions effective to bind hsp90 and form an hsp90-polyphenol complex, wherein the complex binds to the aryl hydrocarbon receptor and modifies expression of one or more genes that are regulated by the aryl hydrocarbon receptor.

Competitive binding assays under numerous conditions optimal for low affinity ligands strongly suggest that EGCG does not bind directly to the AhR. In fact, the present invention relates to a ligand-independent mechanism of antagonist action involving direct binding to the chaperone protein hsp90. This binding of EGCG to hsp90 results in nuclear localization of an AhR form incapable of binding to DNA, supporting a model in which the AhR is translocated to the nucleus in the presence of hsp90. This mechanism therefore provides a useful screening tool to identify potential chemotherapeutic agents.

### BRIEF DESCRIPTION OF THE DRAWINGS

FIGS. 1A-B demonstrate that EGCG inhibits TCDD-mediated gene induction in both mouse and human hepatoma cells. FIG. 1A shows the results of a Western blot on Hepa1c1c7 cells that were treated for the indicated time with either DMSO (D), 150 pM TCDD (T), 200 μM EGCG alone (E), or 200 μM EGCG in the presence of 150 pM TCDD (E+T). Proteins were separated by SDS-PAGE and blotted for CYP1A1 and actin as a loading control. FIG. 1B is graph illustrating the effect of TCDD on luciferase reporter gene expression. HepG2.101L cells were treated with 500 pM TCDD and increasing concentrations of EGCG for 4 h (n=4). Values are presented as percent of observed luciferase induction in the presence of 500 pM TCDD alone ±SD. Representative data from one of at least three separate experiments are shown.

FIGS. 2A-B demonstrate that EGCG does not compete for binding to the AhR ligand binding site. Competitive binding was analyzed by velocity sedimentation on 10-30% sucrose gradients in a vertical tube rotor. Hepa cytosol was treated with 3 nM ³H-TCDD (FIG. 2A) or 5 nM ³H-BNF (FIG. 2B) in the presence of the indicated compound for 2 h at room temperature. Cytosols were loaded onto sucrose gradients, spun, and fractionated. Fractions were analyzed for the presence of ³H, indicative of a ligand-bound AhR complex. The above data are representative of at least three experiments.

FIG. 3 illustrates, by immunoblotting, the co-elution of hsp90 and XAP2 from EGCG conjugated beads. ³⁵S-AhR, -ARNT, -p23, and -XAP2 were synthesized in vitro in rabbit reticulocyte lysate (“RRL”). Unconjugated CNBr-activated Sepharose (U), or EGCG-conjugated Sepharose (C) were incubated with either RRL alone or TnT translated ³⁵S-AhR, -ARNT, -p23, or -XAP2 in RRL. The beads were washed and bound protein eluted. Total eluted protein was separated by SDS-PAGE. Input lysate (54) was loaded as a control (ctl). ³⁵S-labeled protein was detected by Phosphoimaging. The hsp90 inherent to RRL was detected by immunoblotting. FIG. 3 is representative of three experiments.

FIG. 4 illustrates, by immunoblotting, that XAP2 indirectly binds EGCG-Sepharose by its direct binding to hsp90. Unconjugated CNBr-activated Sepharose (U), or EGCG-conjugated Sepharose (C) were incubated with either purified hsp90 (hsp-(P)), purified ³⁵S-XAP (His-XAP2), His-XAP2 in the presence of purified hsp90, or His-XAP2 in RRL. The beads were washed and bound protein eluted. Total eluted protein was separated by SDS-PAGE. Input lysate (54) was loaded as a control (ctl). XAP2 was visualized by Phosphoimaging and hsp90 visualized by immunoblotting. FIG. 4 is representative of three experiments.

FIG. 5 is a map of full-length chicken hsp90, single amino acid mutants, or truncation mutants and their ability to bind EGCG-conjugated Sepharose. The top panel illustrates various mutant hsp90 constructs. Wild type (wt) chicken hsp90 consists of 728 amino acids. Various mutated amino acids are marked by an X. In the bottom panels, RRL containing the indicated in vitro transcribed ³⁵S-hsp90 construct was incubated with either unconjugated (U), or EGCG-conjugated (C) Sepharose. The beads were washed and bound protein eluted. Total eluted protein was separated by SDS-PAGE. Input lysate (54) was loaded as a control (ctl). Hsp90 was visualized by phosphoimaging. All truncation mutants were run on the same gel. The lower signal associated with the smaller fragments required additional grayscale image adjustments and therefore appear as a separate image. FIG. 5 is representative of three experiments.

FIG. 6 illustrates the ability of EGCG to induce nuclear localization of the AhR. Hepa cells were treated for 1 h with DMSO, 150 pM TCDD, 200 μM EGCG alone, or 200 μM EGCG in the presence of 150 pM TCDD. Cells were stained with anti-AhR and visualized with Alexa Fluor conjugated fluorescent secondary antibody (middle panels). To help visualize nuclear localization, nuclei were detected with DAPI and overlaid with the AhR image (right panels). Nuclear localization is emphasized by the absence of blue staining and the presence of purple staining within the nuclear compartment of the cell. The images depicted in FIG. 6 are representative of three experiments.

FIG. 7 illustrates the ability of EGCG to inhibit TCDD-induced DRE binding. Hepa cytosol was treated with the indicated concentrations of EGCG in the absence or presence of 3 nM TCDD for 2 h. Treated cytosols were incubated with ³²P-DRE and levels of transformed DRE-AhR complex determined as described in the examples. The audioradiograms illustrated in FIG. 7 are representative of three experiments.

FIG. 8 illustrates that EGCG does not affect AhR degradation. Hepa cells were treated for the indicated time with either DMSO (D), 150 pM TCDD (T), 200 μM EGCG (E), or 200 μM EGCG in the presence of 150 pM TCDD (E+T). Proteins were separated by SDS-PAGE and blotted for AhR (top) and actin (bottom) as a loading control. The western blots shown in FIG. 8 are representative of three experiments.

FIG. 9 demonstrates via western blot that EGCG treatment results in an AhR complex that differs from both the latent and TCDD-activated complex. Hepa cytosol was treated for 2 h with DMSO (D), 10 nM TCDD (T), or 200 μM EGCG (E) and loaded onto a 10-30% sucrose density gradient for analysis by velocity sedimentation in a vertical tube rotor. The gradients were fractionated, and the presence of the AhR within each fraction assessed by western blotting. ¹⁴C-BSA (4.4S) was used as a sedimentation standard and was detected in fraction 7 as indicated by the asterisk. The AhR was not detected in fractions 1-5 under any treatment conditions. The western blots shown in FIG. 9 are representative of three experiments.

FIG. 10 demonstrates via western blot that EGCG alters hsp90 complex association as assessed by density sedimentation. Hepa cytosol was treated for 2 h with DMSO (D), 10 nM TCDD (T), or 200 μM EGCG (E) and loaded onto a 10-30% sucrose density gradient for analysis by velocity sedimentation in a vertical tube rotor. The gradients were fractionated and the presence of hsp90 within each fraction was assessed by western blotting. ¹⁴C-BSA (4.4S) was used as a sedimentation standard and was detected in fraction 7 as indicated by the asterisk. Hsp90 was not detected in fractions 1-5 or 16-24 under any treatment conditions. The western blots shown in FIG. 10 are representative of three experiments.

FIG. 11 is a schematic illustration of a proposed model for the dynamic complex association of the AhR. Following assembly of the AhR-hsp90 complex the AhR exists in multiple forms within the cell determined by the absence/presence of XAP2 and/or p23. These receptor forms exist in a dynamic equilibrium with one another and it is possible that they are functionally unique. Ligand binding to the AhR (such as TCDD) exposes the nuclear localization signal (NLS) and a proposed degradation signal (DS). However this conformational change is not sufficient for DNA binding, and transformation to an AhR-ARNT conformation requires an additional currently undefined event. Binding of EGCG to hsp90 alters the AhR conformation to expose the NLS and shifts the equilibrium towards an AhR complex associated with XAP2 and void of p23. The inhibitory effect of EGCG on AhR transformation involves recruitment of an additional unknown protein to the AhR complex (represented by the dashed hexagon). The presence of TCDD has its own influence on AhR conformation, exposing the DS. Yet, the EGCG-induced conformation prevents TCDD mediated AhR-ARNT association either through stabilization of the AhR-hsp90 interaction, or through prevention of the currently undefined transformation event.

FIG. 12 illustrates the results of an SDS-PAGE separation experiment showing the effect of EGCG on AhR/Arnt complex formation. AhR and Arnt were separately in vitro translated using the TNT RRL system. For each experiment, only one of them translated in the presence of [³⁵S]Methionine. Equal volumes of diluted AhR and Arnt translation were mixed, incubated with D (DMSO), T (1 nM TCDD), E (200 μM EGCG), or T plus E and immunoprecipitated with anti-AhR antibody. All samples were separated by 7.5% SDS-PAGE, transferred to PVDF membrane, and visualized by phosphorlmager.

FIG. 13 illustrates the results of an SDS-PAGE separation experiment showing the interaction of EGCG with hsp90. Chicken hsp90 was translated in vitro using the TNT RRL system in the presence of [³⁵S]Methionine, diluted, incubated with DMSO or EGCG, and then treated with trypsin at indicated concentrations for 10 min at room temperature. All samples were separated by 10% SDS-PAGE, transferred to PVDF membrane, and visualized by phosphorImager. This experiment was performed in the absence of AhR or Amt.

### DETAILED DESCRIPTION OF THE INVENTION

One aspect of the present invention relates to a method of screening compounds for binding to hsp90. This method involves the steps of: exposing a compound to hsp90 or a polypeptide fragment thereof comprising an ATP-binding site; and determining whether the compound binds to hsp90 or the polypeptide fragment thereof.

Hsp90 can be any mammalian hsp90, preferably from a cow, horse, pig, sheep, goat, dog, cat, rabbit, rodent, non-human primate, or human. Exemplary mammalian hsp90 proteins are reported at Genbank Accession Nos. NM_{—}001017963 (human), NM_{—}010480 and NM_{—}008302 (mouse), NM_{—}213973 (pig), AY695393 (rat), AY383484 (horse), and AF548366 (goat) (partial sequence only), which are hereby incorporated by reference in their entirety.

Human hsp90 has a nucleotide sequence corresponding to SEQ ID NO: 1, as follows:

| | |
|---|---|
| gggtgtggcc tccgggcggc atggctgctt ctcaggtgat gccggcttca gctagtgggg | 60 |
| tctagttgac cgttccgcag ccgccagggc cagcggaaag ccggtcaggg ggaaccgcgg | 120 |
| cggggctggt gtcatgagcc tgaggtgaac ttgagggtgc ctcctcagcg gtctcccgcc | 180 |
| ctgccctgag gggcgccggg accccaaaga gcggaggaag agcgccaccc cgacggccac | 240 |
| cgcttcggag ccagcacgcg gggtacccta cggggagcgc ggatgccccc gtgttcgggc | 300 |
| ggggacggct ccacccctcc tgggccctcc cttcgggaca gggactgtcc cgcccagagt | 360 |
| gctgaatacc cgcgcgaccg tctggatccc cgcccaggaa gcccctctga agcctcctcg | 420 |
| ccgccgtttc tgagaagcag ggcacctgtt aactggtacc aagaaaaggc ccaagtgttt | 480 |
| ctctggcatc tgttggtgtc tggatccacc actctactct gtctctggaa acagcccttc | 540 |
| cacgtctctg cattccctgt cactgcgtca ctggccttca gacagagcca aggtgcaggg | 600 |
| caacacctct acaaggatct gcagccattt atattgctta ggctactgat gcctgaggaa | 660 |
| acccagaccc aagaccaacc gatggaggag gaggaggttg agacgttcgc ctttcaggca | 720 |
| gaaattgccc agttgatgtc attgatcatc aatactttct actcgaacaa agagatcttt | 780 |
| ctgagagagc tcatttcaaa ctcatcagat gcattggaca aaatccggta tgaaagcttg | 840 |
| acagatccca gtaaattaga ctctgggaga gagctgcata ttaaccttat accgaacaaa | 900 |
| caaggtcgaa ctctcactat tgtggatact ggaattggaa tgaccaaggc tgacttgatc | 960 |
| aataaccttg gtactatcgc caagtctggg accaaagcgt tcatggaagc tttgcaggct | 1020 |
| ggtgcagata tctctatgat tggccagttc ggtgttggtt tttattctgc ttatttggtt | 1080 |
| gctgagaaag taactgtgat caccaaacat aacgatgatg agcagtacgc ttgggagtcc | 1140 |
| tcagcagggg gatcattcac agtgaggaca gacacaggtg aacctatggg tcgtggaaca | 1200 |
| aaagttatcc tacacctgaa agaagaccaa actgagtact tggaggaacg aagaataaag | 1260 |
| gagattgtga agaaacattc tcagtttatt ggatatccca ttactctttt tgtggagaag | 1320 |
| gaacgtgata aagaagtaag cgatgatgag gctgaagaaa aggaagacaa agaagaagaa | 1380 |
| aaagaaaaag aagagaaaga gtcggaagac aaacctgaaa ttgaagatgt tggttctgat | 1440 |
| gaggaagaag aaaagaagga tggtgacaag aagaagaaga agaagattaa ggaaaagtac | 1500 |
| atcgatcaag aagagctcaa caaaacaaag cccatctgga ccagaaatcc cgacgatatt | 1560 |
| actaatgagg agtacggaga attctataag agcttgacca atgactggga agatcacttg | 1620 |
| gcagtgaagc atttttcagt tgaaggacag ttggaattca gagcccttct atttgtccca | 1680 |
| cgacgtgctc cttttgatct gtttgaaaac agaaagaaaa agaacaatat caaattgtat | 1740 |
| gtacgcagag ttttcatcat ggataactgt gaggagctaa tccctgaata tctgaacttc | 1800 |
| attagagggg tggtagactc ggaggatctc cctctaaaca tatcccgtga gatgttgcaa | 1860 |
| caaagcaaaa ttttgaaagt tatcaggaag aatttggtca aaaaatgctt agaactcttt | 1920 |
| actgaactgg cggaagataa agagaactac aagaaattct atgagcagtt ctctaaaaac | 1980 |
| ataaagcttg gaatacacga agactctcaa aatcggaaga agctttcaga gctgttaagg | 2040 |
| tactacacat ctgcctctgg tgatgagatg gtttctctca aggactactg caccagaatg | 2100 |
| aaggagaacc agaaacatat ctattatatc acaggtgaga ccaaggacca ggtagctaac | 2160 |
| tcagcctttg tggaacgtct tcggaaacat ggcttagaag tgatctatat gattgagccc | 2220 |
| attgatgagt actgtgtcca acagctgaag gaatttgagg ggaagacttt agtgtcagtc | 2280 |
| accaaagaag gcctggaact tccagaggat gaagaagaga aaaagaagca ggaagagaaa | 2340 |
| aaaacaaagt ttgagaacct ctgcaaaatc atgaaagaca tattggagaa aaaagttgaa | 2400 |
| aaggtggttg tgtcaaaccg attggtgaca tctccatgct gtattgtcac aagcacatat | 2460 |
| ggctggacag caaacatgga gagaatcatg aaagctcaag ccctaagaga caactcaaca | 2520 |
| atgggttaca tggcagcaaa gaaacacctg gagataaacc ctgaccattc cattattgag | 2580 |
| accttaaggc aaaaggcaga ggctgataag aacgacaagt ctgtgaagga tctggtcatc | 2640 |
| ttgctttatg aaactgcgct cctgtcttct ggcttcagtc tggaagatcc ccagacacat | 2700 |
| gctaacagga tctacaggat gatcaaactt ggtctgggta ttgatgaaga tgaccctact | 2760 |
| gctgatgata ccagtgctgc tgtaactgaa gaaatgccac cccttgaagg agatgacgac | 2820 |
| acatcacgca tggaagaagt agactaatct ctggctgagg gatgacttac ctgttcagta | 2880 |
| ctctacaatt cctctgataa tatattttca aggatgtttt tctttatttt tgttaatatt | 2940 |
| aaaaagtctg tatggcatga caactacttt aaggggaaga taagatttct gtctactaag | 3000 |
| tgatgctgtg ataccttagg cactaaagca gagctagtaa tgctttttga gtttcatgtt | 3060 |
| ggtttatttt cacagattgg ggtaacgtgc actgtaagac gtatgtaaca tgatgttaac | 3120 |
| tttgtgtggt ctaaagtgtt tagctgtcaa gccggatgcc taagtagacc aaatcttgtt | 3180 |
| attgaagtgt tctgagctgt atcttgatgt ttagaaaagt attcgttaca tcttgtagga | 3240 |
| tctacttttt gaacttttca ttccctgtag ttgacaattc tgcatgtact agtcctctag | 3300 |
| aaataggtta aactgaagca acttgatgga aggatctctc cacagggctt gttttccaaa | 3360 |
| gaaaagtatt gtttggagga gcaaagttaa aagcctacct aagcatatcg taaagctgtt | 3420 |
| caaaaataac tcagacccag tcttgtggat ggaaatgtag tgctcgagtc acattctgct | 3480 |
| taaagttgta acaaatacag atgagttaaa ag | 3512 |

Human hsp90 protein has an amino acid sequence corresponding to SEQ ID NO: 2, as follows:

| | |
|---|---|
| MPPCSGGDGS TPPGPSLRDR DCPAQSAEYP RDRLDPRPGS PSEASSPPFL RSRAPVNWYQ | 60 |
| EKAQVFLWHL LVSGSTTLLC LWKQPFHVSA FPVTASLAFR QSQGAGQHLY KDLQPFILLR | 120 |
| LLMPEETQTQ DQPMEEEEVE TFAFQAEIAQ LMSLIINTFY SNKEIFLREL ISNSSDALDK | 180 |
| IRYESLTDPS KLDSGRELHI NLIPNKQGRT LTIVDTGIGM TKADLINNLG TIAKSGTKAF | 240 |
| MEALQAGADI SMIGQFGVGF YSAYLVAEKV TVITKHNDDE QYAWESSAGG SFTVRTDTGE | 300 |
| PMGRGTKVIL HLKEDQTEYL EERRIKEIVK KHSQFIGYPI TLFVEKERDK EVSDDEAEEK | 360 |
| EDKEEEKEKE EKESEDKPEI EDVGSDEEEE KKDGDKKKKK KIKEKYIDQE ELNKTKPIWT | 420 |
| RNPDDITNEE YGEFYKSLTN DWEDHLAVKH FSVEGQLEFR ALLFVPRRAP FDLFENRKKK | 480 |
| NNIKLYVRRV FIMDNCEELI PEYLNFIRGV VDSEDLPLNI SREMLQQSKI LKVIRKNLVK | 540 |
| KCLELFTELA EDKENYKKFY EQFSKNIKLG IHEDSQNRKK LSELLRYYTS ASGDEMVSLK | 600 |
| DYCTRMKENQ KHIYYITGET KDQVANSAFV ERLRKHGLEV IYMIEPIDEY CVQQLKEFEG | 660 |
| KTLVSVTKEG LELPEDEEEK KKQEEKKTKF ENLCKIMKDI LEKKVEKVVV SNRLVTSPCC | 720 |
| IVTSTYGWTA NMERIMKAQA LRDNSTMGYM AAKKHLEINP DHSIIETLRQ KAEADKNDKS | 780 |
| VKDLVILLYE TALLSSGFSL EDPQTHANRI YRMIKLGLGI DEDDPTADDT SAAVTEEMPP | 840 |
| LEGDDDTSRM EEVD | 854 |

Polypeptide fragments of hsp90 are preferably from human hsp90 of SEQ ID NO: 2. An exemplary fragment of human hsp90 is one containing amino acid residues 538-728 of the full length hsp90. Alternatively, the fragment can be corresponding amino acid residues from any of the other known or subsequently identified mammalian hsp90 protein as determined, for example, by any known sequence alignment algorithm, such as BLAST.

Basically, either the compound or the protein or polypeptide is bound to a substrate, and detection of binding is confirmed by detecting, respectively, presence of the bound protein or polypeptide or presence of the bound compound in any eluent obtained after elution from the substrate. Depending on exactly what is being detected in the eluent, any suitable detection scheme can be utilized. For detection of the compound, mass spectrometry or other detection procedures suitable for detection of small molecules can be utilized. For detection of the hsp90 protein or polypeptide, any suitable immunoassay using polyclonal or monoclonal antibodies (or binding fragments thereof) specific for the antigen can be utilized.

Exemplary immunoassays include, without limitation, enzyme-linked immunoabsorbent assay, radioimmunoassay, gel diffusion precipitin reaction assay, immunodiffusion assay, agglutination assay, fluorescent immunoassay, protein A immunoassay, or immunoelectrophoresis assay.

Monoclonal antibody production may be effected by techniques which are well-known in the art. Basically, the process involves first obtaining immune cells (lymphocytes) from the spleen of a mammal (e.g., mouse) which has been previously immunized with the antigen of interest either in vivo or in vitro, in this case hsp90 or a polypeptide fragment thereof. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature, 256:495 (1975), which is hereby incorporated by reference.

Mammalian lymphocytes are immunized by in vivo immunization of the animal (e.g., a mouse) with the hsp90 protein or polypeptide fragment thereof. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents. (See Milstein and Kohler, Eur. J. Immunol., 6:511 (1976), which is hereby incorporated by reference.) This immortal cell line, which is preferably murine, but may also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the hsp90 protein or fragment thereof subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized with pentobarbital 150 mg/Kg IV. This and other procedures for raising polyclonal antibodies are disclosed in Harlow et. al., editors, Antibodies: A Laboratory Manual (1988), which is hereby incorporated by reference in its entirety. If desired, the polyclonal antibodies can be treated to remove non-specific antibodies, thereby rendering the polyclonal serum mono-specific for a single target. This can be carried out via known procedures.

Another aspect of the present invention relates to a method of screening compounds for inhibition of hsp90 activity. This method involves the steps of contacting a cell with a compound that induces AhR-regulated gene expression and a test compound that binds to hsp90 (e.g., in a region containing the ATP-binding site) or has otherwise been identified by the above-noted method of screening for hsp90 binding activity; and then determining whether, in the presence of hsp90, said contacting is effective to inhibit AhR-induced transcription of a gene containing a dioxin response element, wherein inhibition of AhR-induced expression of the gene indicates the test compound can inhibit hsp90 activity required for AhR-induced transcription.

The cell can be any in vitro cell line or any ex vivo isolated cell. The cell line is preferably a mammalian cell line. Mammalian cells suitable for carrying out the present invention include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, and NS-1 cells. Particularly preferred cell lines include, without limitation, mouse and human hepatoma cell lines.

For the various screening procedures that utilize gene expression as an indicator of activity or inhibition of activity, e.g., of AhR, the exemplary genes which are transcriptionally regulated by the (activated) aryl hydrocarbon receptor are described infra. In one embodiment, the gene may be endogenous to the cell. In an alternative embodiment, the gene is a recombinant reporter gene contained in a recombinant host cell.

Expression of recombinant genes can be carried out by introducing a nucleic acid molecule encoding the gene into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5′→3′) orientation and correct reading frame. The vector contains the necessary elements for the transcription and translation of the inserted protein-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Suitable expression vectors for directing expression in mammalian cells generally include a promoter, as well as other transcription and translation control sequences known in the art. Common promoters include SV40, MMTV, metallothionein-1, adenovirus Ela, CMV, immediate early, immunoglobulin heavy chain promoter and enhancer, and RSV-LTR.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Depending on the vector system and host utilized, any number of suitable transcription and/or translation elements, including constitutive, inducible, and repressible promoters, as well as minimal 5′ promoter elements may be used. In the embodiments of the present invention, where screening occurs, it is preferred that the endogenous or recombinant reporter gene contains an inducible promoter that contains a dioxin response element (i.e., and therefore is inducible by an active AhR).

The protein-encoding nucleic acid, a promoter molecule of choice, a suitable 3′ regulatory region and, if desired, a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

Once the isolated nucleic acid molecule has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, insect cells, plant cells, and the like.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes” that encode enzymes providing for production of an identifiable compound, or other markers that indicate relevant information regarding the outcome of gene delivery, are suitable. For example, various luminescent or phosphorescent reporter genes are also appropriate, such that the presence of the heterologous gene may be ascertained visually.

A further aspect of the present invention relates to a method of screening compounds as a cancer therapeutic. Basically, having identified a compound as an inhibitor of hsp90, via demonstrating that the compound is effective to inhibit AhR-induced transcription of a gene containing a dioxin response element, the inhibition of AhR-induced expression of the gene further indicates the compound is a potential cancer therapeutic. The compound can then be screened against specific cancer cell lines via in vitro testing and in vivo (induced tumor) animal models.

Thus, another aspect of the present invention relates to a method of treating a cancerous condition that includes the step of inhibiting an interaction between hsp90 and a protein that is a causative agent of a cancerous condition, whereby said inhibiting modifies the activity of the protein that is a causative agent of the cancerous condition and thereby treats the cancerous condition. In this embodiment, the inhibition of hsp90 activity or interaction with another protein (that is a causative agent of cancer) can be achieved using the polyphenols (e.g., catechin compounds) described below, but more preferably catechin derivatives obtained by modified substituents of the catechin ring systems, and even more preferably any compounds identified by the above-described screening approaches. In another embodiment, the polyphenol is a flavanol other than a catechin.

“Treating cancerous conditions” specifically refers to administering therapeutic agents to a patient diagnosed of cancer, i.e., having established cancer in the patient, to inhibit the further growth or spread of the malignant cells in the cancerous tissue, and/or to cause the death of the malignant cells. In particular, but without limitation, solid tumors such as breast cancers, colon cancers, prostate cancers, lung cancers, skin cancers, and lymphoid cancers may be amenable to the treatment by the methods of the present invention. “Treating cancerous conditions” also encompasses treating a patient having premalignant conditions to stop the progression of, or cause regression of, the premalignant conditions. Examples of premalignant conditions include hyperplasia, dysplasia, and metaplasia.

Treating cancerous conditions involves treating cells (e.g., cancer cells), preferably in vivo. For therapeutic purposes, polyphenols or other compounds used for inhibition of hsp90 activity or AhR activity are delivered into the cancerous cell in a manner which affords the polyphenol or other compound to be active within the cell. A number of known delivery techniques can be utilized for the delivery, into cells, of polyphenols or other compounds used for inhibition of hsp90 activity or AhR activity.

In accordance with any aspect of the present invention, the polyphenols or other compounds used for inhibition of hsp90 activity or AhR activity can be used alone or in combination with other compounds that can similarly inactivate hsp90 or AhR. The compounds can be present in any suitable pharmaceutical composition containing suitable carriers, diluents, or adjuvants, with the composition being in a solid or liquid form suitable for administration orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes, such as, that of the nose, throat, and bronchial tubes (i.e., inhalation).

One approach for delivering polyphenols or other compounds used for inhibition of hsp90 activity or AhR activity into cells involves the use of liposomes. Basically, this involves providing a liposome which includes that polyphenol or other compound to be delivered, and then contacting the target cell with the liposome under conditions effective for delivery of the polyphenol or other compound into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which are hereby incorporated by reference in their entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

An alternative approach for delivery of polyphenols or other compounds used for inhibition of hsp90 activity or AhR activity involves the conjugation of the desired polyphenol or other compound to a polymer that is stabilized to avoid enzymatic degradation of the conjugated polyphenol or other compound.

Micellar systems formed from block copolymers can also be used to deliver polyphenols or other compounds used for inhibition of hsp90 activity or AhR activity (Kabanov et al., “Micelles of Amphiphilic Block Copolymers as Vehicles for Drug Delivery,” In Amphiphilic Block Copolymers: Self-Assembly and Applications, edited by Alexamdris et al., Netherlands; Kwon et al., J. Controlled Release 48:195-201 (1997); La et al., Journal of Pharmaceutical Sciences 85:85-90 (1996); Kataoka et al., J. Control. Release 24:119-132 (1992); and Bader et al., Angewandte Makromolekulare Chemie 123:457-485 (1984), which are hereby incorporated by reference in their entirety).

Micelles are formed from individual block copolymer molecules, each of which contains a hydrophobic block and a hydrophilic block. The amphiphilic nature of the block copolymers enables them to self-assemble to form nanosized aggregates of various morphologies in aqueous solution such that the hydrophobic blocks form the core of the micelle, which is surrounded by the hydrophilic blocks, which form the outer shell (Zhang et al., Science 268:1728-1731 (1995); Zhang et al., Science 272:1777-1779 (1996), which are hereby incorporated by reference in its entirety). The inner core of the micelle creates a hydrophobic microenvironment for the non-polar therapeutic agent, while the hydrophilic shell provides a stabilizing interface between the micelle core and the aqueous medium. The properties of the hydrophilic shell can be adjusted to both maximize biocompatibility and avoid reticuloendothelial system uptake.

The size of micelles is usually between 10 nm and 100 nm. This size is small enough to allow access to small capillaries while avoiding reticuloendothelial system uptake. Micelles in this size range are also large enough to escape renal filtration, which increases their blood circulation time.

Yet another aspect of the present invention relates to inhibiting transcription-inducing activity of an aryl hydrocarbon receptor as well as modifying expression of a gene that is activated by an aryl hydrocarbon receptor. These aspects of the present invention are carried out indirectly via hsp90. This aspect can be carried out on cells in vivo or in vitro.

According to one approach, these aspects of the present invention are carried out by contacting a cell with a polyphenol under conditions effective to bind hsp90 (i.e., within the cytosol) and form an hsp90-polyphenol complex, wherein the complex binds to the aryl hydrocarbon receptor and inhibits transcription-inducing activity of the aryl hydrocarbon receptor in the cell (i.e., after translocation of the complex into the nucleus).

Suitable polyphenols include, without limitation, flavondiols, flavonoids, phenolic acids, and flavonols. Flavonols are a group of compounds which include catechins. In one embodiment of the present invention, the polyphenol is a catechin selected from the group consisting of epicatechin (“EC”) epigallocatechin gallate (“EGCG”), gallocatechin (“GC”), epicatechin gallate (“ECG”), and epigallocatechin (“EGC”), as well as combinations thereof and derivatives thereof. In another embodiment, the polyphenol is a flavonol other than a catechin.

The amount of polyphenol to be used for contacting the cell is preferably that which results in an intracellular concentration of the polyphenol that can partially inhibit AhR activity by at least about 50 percent, more preferably at least about 60 percent, 70 percent, or 80 percent, most preferably at least about 90 percent. In certain embodiments, the intracellular concentration of the polyphenol can be sufficient to substantially inhibit AhR-induced transcription (that is, greater than 95 percent inhibition) or nearly completely inhibit AhR-induced transcription (that is, greater than 98 percent inhibition). Unless a continuous supply of the polyphenol is utilized for contacting the cell, it is expected that the exact degree of such inhibition will likewise vary over time.

The hsp90-polyphenol complex that is formed is the result of binding of the C-terminal region of hsp90 by the polyphenol compound, in a region coincident with the C-terminal ATP binding site (i.e., between residues 538 and 728 of the human hsp90 protein (SEQ ID NO: 2).

Without being bound by belief, it is believed that the binding between the polyphenol and hsp90 inhibits release of hsp90 from the aryl hydrocarbon receptor. In other words, binding of the aryl hydrocarbon receptor by the polyphenol-hsp90 complex stabilizes the aryl hydrocarbon receptor within a conformation substantially incapable of binding to a dioxin-response element.

A number of genes possess dioxin-response element and are therefore transcriptionally regulated by the (activated) aryl hydrocarbon receptor. Genes that are regulated by the activated aryl hydrocarbon receptor include genes that are normally inhibited or downregulated, as well as genes that are activated or upregulated. Exemplary genes include, without limitation, pS2, cathepsin D, Sp1, heat shock protein 27, T cadherin, latent transforming growth factor-β binding protein 1, aryl hydrocarbon receptor repressor (AhRR), NAD(P)H-menadione oxidoreductase 1, plasminogen activator inhibitor-2, ecto-ATPase, interleukin-2, cyclooxygenase-2, UDP glucuronosyltransferase 1, glutathione-S-transferase Ya, CYP1A1, plasminogen activator inhibitor-1, CYP1B1, aldehyde dehydrogenase 4, hairy and enhancer of Split homolog-1 (HES-1), CYP1A2, paraoxonase, proopiomelanocortin (ACTH precursor), c-myc, transforming growth factor-beta, interleukin-6, interferon-gamma, poly(ADP-ribose) polymerase, BSAP, Bax, polκ, DIF-3, Cu/Zn superoxide dismutase, CYP2S1, steroidogenic acute regulatory protein, RANTES, MHC Q1, transforming growth factor-alpha (TGF-α), urokinase plasminogen activator, Interleukin-1β, c-fos, c-jun, ADP ribosylation factor 4, basic transcription factor 2 (34-kDa subunit), cadherin 2, CDC-like kinase, complement component 5, cyclin-dependent kinase inhibitor 1A, cyclin-dependent kinase 1, CYP19A1, DNA mismatch repair protein, early growth response protein, 110-kDa heat-shock protein, heat shock factor-binding protein 1, 60-kDa heat shock protein, insulin-like growth factor-binding protein 10, insulin-like growth factor binding protein 1, insulin-like growth factor II, integrin β, interleukin 1 receptor type 1, 45-kDa interleukin enhancer-binding factor 2, NEDD5 protein homolog, Niemann-Pick C disease protein, retinoblastoma-binding protein 3, Rab geranylgeranyl transferase β subunit, RNA polymerase II elongation factor SIII p15 subunit, Sec61-γ; sex-determining region Y box-containing gene 9, short/branched chain-specific acyl-CoA dehydrogenase, solute carrier family 2 member 2, T-complex protein 1 τ and δ subunits, thyroid receptor-interacting protein 15, topoisomerase I and II α, transcription factor HTF4, translation initiation factor 4E 25-kDa subunit, CYP2C11, albumin, ATP synthetase β subunit, calreticulin precursor, cytochrome B5, CYP2D4, 25DX, endoplasmic reticulum protein ERP29 precursor, ferritin light chain, 78 kDa glucose-regulated protein precursor, glutamate dehydrogenase, glyceraldehydes-3-phosphate dehydrogenase, heat shock protein 72, 3-α-hydroxysteroid dehydrogenase, IκB kinase 2, 150 kDa iodothyronine 5′ monodeiodinase, isocitrate dehydrogenase, oxygen-regulated protein, peroxiredoxin IV, prohibitin, protein disulfide isomerase ER60 precursor, Bcl-2 family genes (bik, bid, Hrk, bok/mtd, mcl-1, bcl-x, and bcl-w), IAP family genes (X-linked IAP, NAIP1, and NAIP5), Myd88, p21, p53, RIP, TNFR, family genes (OX40, Fas, CD30, Ltβ-R, and TNFR1), TNF family genes (LIGHT, OX40L, and Bar-like), TRAF2, lecithin:retinol acyltransferase, actin α, Ahr, alcohol dehydrogenase 1 complex, angiopoietin-like 4, angiotensinogen, brain derived neurotrophic factor, cadherin 16, calbindin-28k, carbonic anhydrase 3, carboxylesterase 3, Cd44 antigen, coagulation factor II, cytokine receptor-like factor 1, epiregulin, fibroblast growth factor 7, fibroblast growth factor receptor 4, follistatin, forkhead box a2 and f2, Fos-like antigen 1, glutamyl aminopeptidase, Gro1 oncogene, high mobility group at-hook 2, α-2-hs-glycoprotein, hydroxysteroid 11-β dehydrogenase 2, insulin-like growth factor 2, insulin-like growth factor binding proteins 3, 5, and 6, integrin α 3, α 6 and β 4, IL-6, interferon activated gene 202a, lymphocyte antigen 6 complex (loci e, A and H), lysyl oxidase, matrix metalloproteinase 3 and 9, mitogen regulated protein proliferin 3, NADH dehydrogenase 1, osteopontin, p21, peripherin, phospholipase a2 group VII, proliferin 2, Ras-related protein, rennin 1 structural, retinol binding protein 4, plasma, RNA binding motif, single stranded interacting protein 1, secreted phosphoprotein 1, small proline-rich proteins 2b, 2c and 2f, spleen tyrosine kinase, squalene epoxidase, stratifin, thrombomodulin, TNF receptor family member 1b, tumor-associated calcium signal transducer 2, ADP-ribosylation-like factor 6 interacting protein 5, calcium binding protein A11, CCAAT/enhancer-binding protein, esterase 10, immediate early response 3, nicotinic acetylcholine receptor subunit α 6, nuclear factor erythroid derived 2, like 2, prenylated SNARE protein, RIKEN-CDNA FLJ13933 FIS, clone Y79AA1000782, RIKEN-phosphogluconate dehydrogenase inhibitor, S100 calcium-binding protein A4, vanin 1, Vomeronasal organ family 2, receptor 11, distal-less homeobox 5, activin receptor type II B, acyl-coenzyme A oxidase, aminoacylase 1, B-cell lymphoma protein 3, basic transcription element binding protein 1, bone morphogenic protein, β-catenin, Cdc42, CDK-2 associated protein, cellular retinoic acid binding protein 1, collagen IV α 3 chain, collagen VI α 3, cyclin-dependent kinase 4 inhibitor C, cyclin-dependent kinase inhibitor 2β iso form, CYP27A1, discoidin receptor tyrosine kinase, E2F dimerization partner 2, early growth response 1, EGF-containing fibulin-like extracellular matrix protein, ephrin A1 (isoform a), epidermal growth factor receptor substrate 15, epithelial-cadherin, fibroblast growth factor, fibronectin receptor β subunit, fos-related protein, GABA A receptor, GATA binding protein 1, glucocorticoid receptor, GTPase activating protein, homospermidine synthase, hsp 70 kDa protein insulin-like growth factor 1 receptor, GABA A receptor ε subunit, 25 kDa GTP binding protein, 1 hsp 70 kDa 2, hyaluronidase 1, insulin induced protein 1, interferon-induced protein 56 and p′78, interferon γ receptor 1, interferon regulatory factor 4, IL-6 receptor β, IL-8, Kruppel-like factor 5, lamanin B2 chain and α 3b chain, leukemia inhibitor factor, low density lipoprotein receptor-related protein, macrophage inflammatory protein 1-β, MAP kinase-activated protein kinase 2, MAP kinase phosphatase-1, matrix metalloproteinase 1 and 9, mesoderm specific transcript iso form, mitotic arrest defective protein, multifunctional DNA repair enzyme, neurotrophic tyrosine kinase, NFκB p100/p49 subunits, nuclear receptor coactivator 2, ornithine cyclodeaminase, 8-oxo-dGTPase, p53, p53-binding protein Mdm4, peripheral benzodiazepine receptor, polyamine oxidase, protein kinase C α, protein kinase C-like 2, protein tyrosine phosphatase type 1, pyruvate dehydrogenase kinase, replication licensing factor, retinoic acid receptor β, RNA polymerase II, S100 calcium binding protein, serine/threonine kinase 4, serine/threonine specific protein phosphatase, serum/glucocorticoid regulated kinase, STAT1, thioltransferase, thioredoxin reductase, thrombin receptor, thrombomodulin, thymosin β 10, tissue inhibitor of metalloproteinase-3, translation initiation factor 3 and 4H, transmembrane 4 superfamily member, tumor-associated calcium signal transducer 4, tyrosine-protein kinase receptor, ubiquitin-like interferon, α-inducible protein, vasoactive intestinal polypeptide receptor, VEGF, vitronectin, WAP four-disulfide core domain 2 (isoform 1) precursor, zinc finger protein 42, DEAD/H box polypeptide 3, DnaJ (hsp40) homolog (subfamily B, member 1), fatty acid binding protein 2 (intestinal), heat shock 70 kDa protein 5, heat shock protein 1α (hsp90), heat shock protein 105, hepatic nuclear factor 4 (HNF4), HIV-tat interactive protein 2, homocysteine-inducible ER stress-inducible ubiquitin-like domain member 1 (Herp), C-type lectin-like receptor 2, lectin (galactose binding, soluble 1), malic enzyme, mannoside acetylglucosaminyltransferase 2, phosphoribosyl pyrophosphate amidotransferase, pleckstrin homology domain containing (family B number 1), Ras homolog gene family member E, ribosomal protein L12, S-100 calcium binding protein A10 (calpactin), signal transducer and activator of transcription 2, solute carrier protein 21 (organic anion transporter, member 10), TNFα-induced adipose-related protein, ubiquitin-specific protease 2, vaccinia related kinase 2, zinc finger protein 191, matrix metalloproteinase-1, CK8 polypeptide, glutathione peroxidase, Ig lambda-1 chain C region, Ig lambda-2 chain C region, angiogenin, Bad, bcl-w (Bcl2-like 2), casper, caspases 1, 3, 7, 8, 11, and 14, CRADD, cyclin-dependent kinase inhibitor p21 Waf1, DAXX (fas-binding protein), DR5 (TRAIL death-inducing receptor), Fas ligand, IAP 1 and 2 (inhibitor of apoptosis proteins 1 and 2), fibroblast growth factor, G-CSF, GADD45 (DNA-damage inducible transcript 1), HGF (hepatocyte growth factor), ILs 3, 4, 5, 6, 7, 9, 10, 12α, 15, and 18, mdm2, NFκb1, NF-κB inducing kinase, p53 responsive protein, PDGFα, retinoblastoma supsceptibility protein, RIP (cell death protein), thrombospondin 3, TNFβ, TRAF2 (TNF receptor associated factor 2), (TRAF3 (death adaptor molecule), TRAF6 (CD40 associated factor), Trail (TNF-related apoptosis inducing ligand), TRIP (TRAF-interacting protein), tumor necrosis factor I and II receptors, and VEGF-B, C, D, and I.

Transcription of other native or non-native genes containing DREs can also be modulated by binding of hsp90 protein with a compound in accordance with the present invention.

### EXAMPLES

The following examples are provided to illustrate embodiments of the present invention but are by no means intended to limit its scope.

### Materials and Methods

### Chemicals

2,3,7,8-tetrachlorodibenzo-p-dioxin (TCDD) was purchased from Cambridge Isotope Laboratories (Cambridge, Mass.). ³H-TCDD (specific activity: 34.7 Ci/mM) was purchased from Chemsyn Science Laboratories (Lenexa, Kans.). EGCG was purchased from Sigma Chemical Company (St. Louis, Mo.). 2,3,7,8-tetrachlorodibenzofuran (TCDF) was a kind gift from Dr. Steven Safe (Texas A&M University, College Station, Tex.). [Methyl-¹⁴C]-bovine serum albumin was purchased from Perkin Elmer Life Sciences, Inc. (Boston, Mass.). 3M4NF and 3′-nitroflavone were synthesized in the laboratory of Dr. Andrew Kende (Dept. of Chemistry, University of Rochester) as previously described (Henry et al., Mol. Pharmacol. 55:716-725 (1999), which is hereby incorporated by reference in its entirety). [3′,5′-³H]-β-Naphthoflavone (³H-BNF) was a kind gift from Dr. Mark Hahn (Woods Hole, Mass.).

Preparation of Cytosol

Mouse hepatoma cells, Hepa1c1c7 (Hepa), and BP^{r}Cl cells were maintained in modified Eagle's medium (Sigma) supplemented with 10% heat-inactivated fetal bovine serum, sodium pyruvate, L-glutamine, sodium bicarbonate, and Gentamicin (MEM+), at 37° C. in a humid atmosphere with 5% CO₂. BP^{r}Cl cells are a derivative of Hepa1c1c7 cells that lack ARNT protein expression and function (Probst et al., Mol. Pharmacol. 44:511-518 (1993), which is hereby incorporated by reference in its entirety). Upon reaching 90% confluency, cells were harvested and homogenized in HEDG buffer (25 mM HEPES, 1.5 mM Na₂EDTA, 1 mM dithiothreitol, 10% (v/v) glycerol, pH 7.6) containing protease inhibitors (Roche Applied Science complete mini cocktail tablet, Indianapolis, Ind.). Both Hepa and BP^{r}C1 cytosols were prepared by centrifugation of the homogenate at 100,000 g for 45 min.

SDS-PAGE and Western Blot Analysis

Hepa cells were plated onto 6-well plates at a density of 5×10⁵ cells/well and incubated overnight at 37° C. Cells were treated with vehicle (DMSO), TCDD (150 pM), EGCG, or EGCG in the presence of TCDD (150 pM) for 4 hrs. Cells were lysed (0.2% Triton, 5 mM EDTA in PBS) and total protein quantified using the Coomassie Plus Protein Assay Reagent (Pierce, Rockford, Ill.). Protein (30 μg) was separated by SDS-PAGE (7.5% acrylamide resolving gel) and transferred to PVDF membrane (Millipore, Bedford, Mass.). Membranes were probed with antibodies recognizing CYP1A1 (Xenotech, Lenexa, Kans.), AhR (Biomol, Plymouth Meeting, Pa.) and actin (Sigma). The secondary IgG antibodies were coupled to horseradish peroxidase (Jackson Immuno Research, West Grove, Pa.). Both primary and secondary antibodies were used at a dilution of 1:5000 in TBST (50 mM Tris, 300 mM NaCl, 0.5% Tween 20, pH 7.5) containing 5% milk. Proteins were visualized by chemiluminescence (KPL, Gaithersburg, Md.).

Luciferase Reporter Gene Assay in Human Cells

The human hepatoma cell line HepG2 were stably transfected with a luciferase expression vector downstream from exon one, a portion of intron one, and 1612 base pairs of the 5′-flanking sequence of the human CYP1A gene to generate the cell line HepG2.101L, as previously described (Postlind et al., Toxicol. Appl. Pharmacol 118:255-262 (1993), which is hereby incorporated by reference in its entirety). Cells were grown in Dulbecco's modified Eagle's medium (Mediatech Herndon, Va.) supplemented with 10% fetal bovine serum and Gentamicin (DMEM) at 37° C. in a humid atmosphere with 5% CO₂. Treatment and reporter gene activity were assessed as previously described (Palermo et al., Chem. Res. Toxicol. 16:865-872 (2003), which is hereby incorporated by reference in its entirety) with the following changes: HepG2.101L cells were added to hydrated Cytodex™ microcarrier beads (Sigma) to achieve 1.5×10⁶ cells per 30 mg beads per 10 ml DMEM. Luminescence was detected using the Packard Lumicount™ (Meriden, Conn.).

Sucrose Density Gradient

Competitive binding of EGCG was analyzed by velocity sedimentation on sucrose gradients in a vertical tube rotor (Tsui et al., Can. J. Physiol. Pharmacol 59:927-931 (1981); Jensen et al., Toxicol. Sci. 64:41-56 (2001), which are hereby incorporated by reference in their entirety). Hepa cytosol (100 μl of 2.5 mg/ml) or BP^{r}Cl cytosol was incubated for two hours at room temperature with either 3 nM ³H-TCDD, 0.5 nM ³H-TCDD, or 5 nM ³H-BNF, in the presence of DMSO, 150-fold excess TCDF, 1 μM 3M4NF, or EGCG. Cytosols were washed with charcoal-dextran (1 mg charcoal/mg protein) in HEDG, and applied to a 10-30% (w/v) sucrose gradient in Beckman Quick-Seal centrifuge tubes (Beckman, Palo Alto, Calif.). [Methyl-¹⁴C]-BSA was used as an internal sedimentation marker. The gradients were centrifuged for 2 h at 372,000×g in a Beckman VTI-80 rotor. Fractions were collected from the top at a rate of 1 ml/min, 0.2 ml/fraction. For competitive binding assays, fractions were assayed for radioactivity with ScintiVerse (Fisher Scientific, Fair Lawn, N.J.). That portion of bound TCDD that could be competed with a 150-fold excess of TCDF, a known high affinity AhR ligand and potent agonist, represents AhR specific TCDD binding. For AhR and hsp90 density analysis, protein contained within an aliquot (200 of each fraction was separated by SDS-PAGE (7.5% acrylamide resolving gel) and transferred to PVDF membranes. Membranes were probed with antibodies recognizing AhR (Biomol) or hsp90 (Stressgen, Victoria, BC Canada). A crude estimate of the sedimentation coefficient for the EGCG shifted AhR complex was calculated by the method of Martin and Ames (Martin et al., J. Biol. Chem. 236:1372-1379 (1961), which is hereby incorporated by reference in its entirety) relative to the ¹⁴C-BSA (4.4S) standard.

Plasmid Constructs

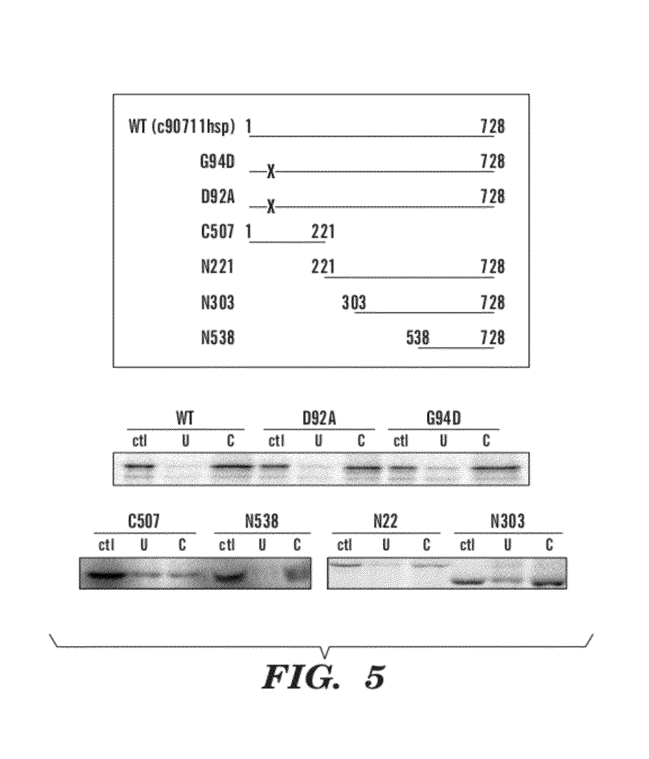

Murine AhR and ARNT cDNA (obtained from J. Whitlock and O. Hankinson, respectively) were inserted into pcDNA3 (Invitrogen, Carlsbad, Calif.). Chicken hsp90 constructs (FIG. 5), c90711hsp (wild-type), c90G94D (wild-type point mutant gly94→Asp (Grenert et al., J. Biol. Chem. 272:23843-23850 (1997), which is hereby incorporated by reference in its entirety), c90D92A (wild-type point mutant Asp92→Ala (Sullivan et al., J. Biol. Chem. 268:20373-20379 (1993), which is hereby incorporated by reference in its entirety), c90N221 (wild-type truncation mutant encoding a.a. 221-728 (Sullivan et al., J. Biol. Chem. 268:20373-20379 (1993), which is hereby incorporated by reference in its entirety)), c90N303 (a.a. 303-728) and c90N538 (a.a. 538-728) were kind gifts from Dr. D. Toft (Rochester, Minn.). The C-terminal truncation mutant c900507 (a.a. 1-221) was obtained from Dr. L. Neckers (Rockville, Md.) (Grenert et al., J. Biol. Chem. 272:23843-23850 (1997); Marcu et al., J. Biol. Chem. 275:37181-37186 (2000), which are hereby incorporated by reference in their entirety). Human p23 cDNA was excised from pQE80-p23 (obtained from W. Chan Stockton, Calif.) by sequential digests using the restriction enzymes Sad (3′-end) and BamHI (5′-end) and inserted into the similarly digested pET28a (Novagen, Madison, Wis.). The mouse histidine-tagged XAP2 construct, pET-AIP (Bell et al., J. Biol. Chem. 275:36407-36414 (2000), which is hereby incorporated by reference in its entirety), was a kind gift from Dr. D. Bell (Nottingham, UK). All constructs are in the T7 orientation with the exception of c900507, which is driven by an SP6 promoter.

In Vitro Transcription/Translation

AhR, ARNT, hsp90 (wild-type, point mutants, and truncation mutants), p23, and XAP2 were generated (separately) by coupled transcription/translation in rabbit reticulocyte lysate (RRL) using the TNT® system according to the manufacturer's protocol (Promega, Madison Wis.). In this system, ³⁵S-methionine was included in the transcription/translation mix to generate ³⁵S-labeled protein.

Immobilized Metal Affinity Chromatography (IMAC) Purification of Histidine-Tagged XAP2. The TALON IMAC method was used for purification of XAP2 from RRL following a modified protocol (Clonetech). A 500 μL (50% slurry) of cobalt resin was washed at room temperature three times with TALON buffer (50 mM Na₂PO₄, 300 mM NaCl, pH 7.0) according to the manufacturer's protocol. A 175 μL aliquot of undiluted ³⁵S-XAP2 in RRL was added to a 15 ml conical tube containing the washed resin in TALON buffer for 20 minutes at room temperature end-over-end. The resin-bound XAP2 was pelleted at 700×g for three minutes and washed with TALON buffer for 15 minutes at room temperature end-over-end. The pelleted resin-bound XAP2 was additionally washed with 7.5 mM imidazole in TALON buffer for 45 min at room temperature end-over-end. The resin-bound XAP2 was then transferred and packed into a two ml disposable column (Pierce) and washed with five column volumes of 7.5 mM imidazole in TALON buffer. Ten 2004 aliquots of 150 mM imidazole in TALON buffer were used to elute the XAP2 from the column. Each aliquot was collected separately and analyzed for the presence of ³⁵S-XAP2 by SDS-PAGE followed by Phosphorimaging (PSI; Molecular Dynamics, Sunnyvale, Calif.).

Affinity Chromatography

EGCG was conjugated to cyanogen bromide (CNBr)-activated Sepharose (Sigma). EGCG (2.5 mg) was dissolved in 5004 coupling buffer (0.1M NaHCO₃, 0.5M NaCl, pH 6.0). CNBr-activated Sepharose was swelled and washed in 1 mM HCl on a sintered glass filter followed by a wash with coupling buffer. CNBr-activated Sepharose beads were added to the EGCG in coupling buffer at a final concentration of 5 mg EGCG/ml of wet gel. The coupling solution containing EGCG and Sepharose was mixed end-over end at 4° C. overnight. Remaining active groups were blocked for 2 h at room temperature in Tris-HCl (0.1M, pH 8). EGCG-conjugated Sepharose was washed with three cycles of alternating pH wash buffers (Buffer 1: 0.1M acetate, 0.5M NaCl, pH4.0; Buffer 2: 0.1M Tris-HCl, 0.5M NaCl, pH 8.0). EGCG-conjugated beads were then equilibrated in binding buffer (0.05M Tris-HCl, 0.15M sodium chloride, pH 7.5). The control unconjugated CNBr-activated Sepharose beads were prepared as above in the absence of EGCG.

Approximately 25 μL of 1:4 diluted RRL containing ³⁵S-labeled in vitro transcribed protein was incubated with 40 μL of either unconjugated or EGCG-conjugated Sepharose beads in binding buffer (50% slurry). For the smallest truncation hsp90 mutants (C507, N538), 50 μL of 1:4 diluted ³⁵S-labeled in vitro transcribed protein was incubated with 80 μL of Sepharose beads to compensate for decreased signal due to fewer methionines. The mixture was diluted with 500 μL binding buffer and incubated end-over-end for one hour at room temperature. The protein-bound Sepharose beads were pelleted by micro-centrifugation at maximum speed for 15 seconds. The beads were washed three times with binding buffer. Bound protein was eluted with SDS-loading buffer (0.125M Tris, 4% SDS (w/v), 20% glycerol (v/v), 200 mM dithiothreitol, 0.01% bromophenol blue (w/v), pH 6.8). The samples were boiled for five minutes and bound protein separated by SDS-PAGE. Protein was transferred to PVDF membrane (Millipore, Bedford, Mass.). ³⁵S-labeled AhR, ARNT, p23, XAP2, and hsp90 truncated and point mutants were detected by Phosphoimaging. Hsp90 inherent to RRL was detected by immunoblotting using an anti-hsp90 monoclonal antibody (Stressgen Victoria, BC Canada) followed by secondary antibody coupled to horseradish peroxidase (Jackson Immuno Research, West Grove, Pa.). Both primary and secondary antibodies were used at a dilution of 1:5000 in TBST.

For affinity chromatography using histidine-purified XAP2 and purified human hsp90 (Stressgen), 30 μl of unconjugated or EGCG-conjugated Sepharose were incubated for 1 h at room temperature with either 1:4 diluted ³⁵S-XAP2 in RRL, 30 μl of histidine purified ³⁵S-XAP2, 0.6 μg of purified hsp90, or 20 μl of histidine purified ³⁵S-XAP2 in the presence of 0.6 μg of purified hsp90. All incubations were adjusted to contain the same concentration of imidazole as the histidine-purified sample. Protein was bound, and beads were washed and eluted as above.

Immunocytochemistry

Hepa cells were plated onto four-well chamber slides (Becton Dickinson, Bedford, Mass.) at a density of 2.0×10⁴ and incubated overnight. Cells were treated with either vehicle (DMSO), TCDD (150 pM), EGCG alone (200 μM), or EGCG in the presence of TCDD (150 pM) for 1 h. Cells were fixed with 3.7% formalin at room temperature for 10 minutes, followed by a four minute incubation in anhydrous methanol at 4° C. All antibodies were filtered through a 0.45 micron filter before staining Cells were blocked for one hour in phosphate buffered saline containing 4% BSA, incubated with anti-AhR at a 1:2000 dilution for two hours at room temperature, followed by a one hour incubation with 1:1000 anti-rabbit Alexa-Fluor conjugated secondary antibody (Molecular Probes, Eugene Oreg.). Nuclei were stained with DAPI (0.5 μg/ml) (Molecular Probes) for three minutes at room temperature. Slides were mounted with 50% glycerol and coverslipped. AhR staining and nuclear staining were visualized using a Nikon Eclipse TS110 fluorescent microscope (40× magnification). Fluorescent images were captured using SPOT advanced software.

Electrophoretic Mobility Shift Assay

Hepa cytosol (2.5 mg protein/ml) was incubated with a range of concentrations of EGCG (1-200 μM) or EGCG in the presence of 3 nM TCDD for 2 h at room temperature. Treated cytosols (21-25 μg) were mixed with nonspecific DNA (herring sperm), 0.08M NaCl, and 25,000-45,000 cpm of [³²P]-endlabeled oligonucleotide containing a single consensus DRE (Gasiewicz et al., Biochem. Pharmacol. 52:1787-1803 (1996), which is hereby incorporated by reference in its entirety). Samples were subjected to nondenaturing electrophoresis (4% acrylamide) and visualized using a Phosphoimager.

### Example 1

### EGCG Inhibits TCDD Induced Gene Expression

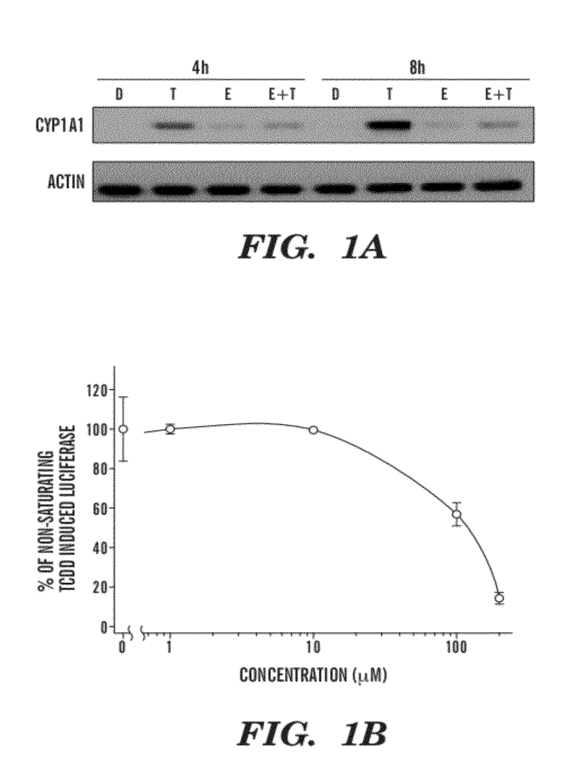

Although it has previously been demonstrated that EGCG alters transcription of a DRE-dependent reporter gene (Palermo et al., Chem. Res. Toxicol. 16:865-872 (2003), which is hereby incorporated by reference in its entirety), it was important to assess the ability of EGCG to influence an endogenous AhR-regulated gene. To do this, the effect of EGCG on CYP1A1 expression in mouse hepatoma cells was determined. CYP1A1 is highly expressed in this cell type and is known to be transcriptionally induced upon ligand activation of the AhR (Whitlock, Annu. Rev. Pharmacol. Toxicol. 39:125 (1999), which is hereby incorporated by reference in its entirety). As shown in FIG. 1A, treatment alone with EGCG had no effect on CYP1A1 gene induction. However, treatment of cells simultaneously with EGCG and TCDD showed a concentration-dependent inhibition of TCDD-mediated CYP1A1 gene induction. These data support the hypothesis that EGCG is an AhR antagonist capable of inhibiting AhR transcription of an endogenous gene.

To determine if this effect could be observed in other cell types, the antagonist activity of EGCG was assessed in the stably transfected human hepatoma cell line, HepG2. The reporter plasmid, described previously (Postlind et al., Toxicol. Appl. Pharmacol 118:255-262 (1993), which is hereby incorporated by reference in its entirety), contains the human CYP1A1 promoter and 5′-flanking sequence upstream of the luciferase gene. In this system, EGCG treatment alone did not induce luciferase activity significantly over background. However, EGCG significantly inhibited TCDD induced luciferase activity (FIG. 1B), with an IC₅₀ value of approximately 100 μM. This IC₅₀ was similar to that observed previously with the mouse cells (Palermo et al., Chem. Res. Toxicol. 16:865-872 (2003), which is hereby incorporated by reference in its entirety). EGCG also inhibited TCDD-mediated CYP1A1 gene induction in HepG2 cells. These data suggest that the antagonist effect of EGCG is not specific for mouse hepatoma cells and that this compound is capable of modulating the activity of the human AhR. The remainder of these experiments elucidate the inhibitory mechanism of EGCG on the mouse AhR.

### Example 2

### EGCG Does Not Compete for Binding to the AhR Ligand Binding Domain

There are many possible mechanisms by which EGCG may function to inhibit TCDD-mediated gene induction. Previous findings suggest that flavonoid antagonists function through direct competition for binding to the TCDD ligand binding site on the AhR (Henry et al., Mol. Pharmacol. 55:716-725 (1999), which is hereby incorporated by reference in its entirety). This binding of antagonist is believed to result in an AhR conformation incapable of nuclear translocation, DRE binding, and transcriptional enhancement. It was therefore hypothesized that EGCG exerts its effects through an identical mechanism involving direct binding to the AhR ligand-binding site.

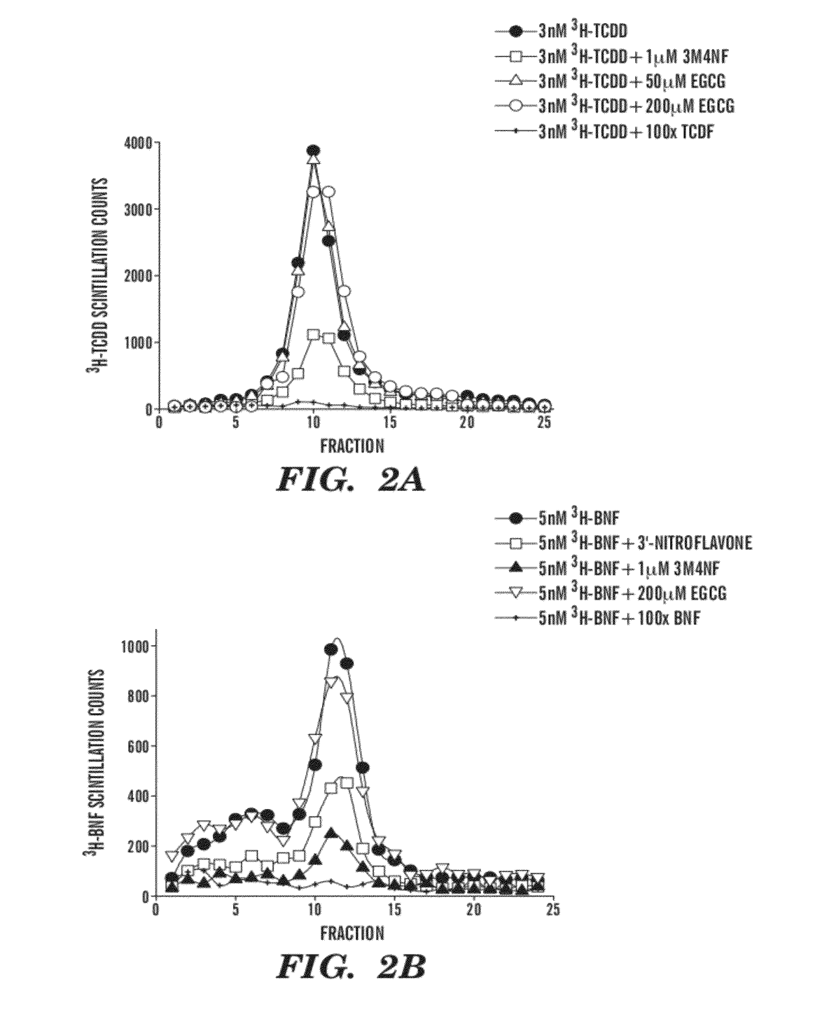

Velocity sedimentation of the AhR on sucrose density gradients was used to determine if EGCG could inhibit the specific binding of TCDD to the mouse AhR. This methodology was chosen over other binding assays because it provides a reliable measure of specific binding to the AhR (Okey et al., J. Biol. Chem. 254:11636-11648 (1979), which is hereby incorporated by reference in its entirety), and has proven successful in detecting binding of many low affinity ligands (Denison et al., Toxicol. Appl. Pharmacol. 152:406-414 (1998); Denison et al., Toxicologist 48:304 (1999), which is hereby incorporated by reference in its entirety). Incubation of Hepa cytosol with ³H-TCDD led to the formation of specifically bound ³H-TCDD-AhR protein complexes within the 9S region (˜fractions 10-15) of the gradient (FIG. 2A). Co-incubation with ³H-TCDD and the known AhR antagonist 3M4NF, inhibited the formation of this ³H-TCDD peak consistent with previous data suggesting that 3M4NF competes for binding to the AhR ligand binding site (Henry et al., Mol. Pharmacol. 55:716-725 (1999), which is hereby incorporated by reference in its entirety). Interestingly, co-incubation with EGCG failed to attenuate the ³H-TCDD 9S signal. These data surprisingly indicate that either EGCG does not bind to the TCDD ligand-binding site or it does so with very low affinity.

Sucrose density gradient experiments were also performed using the lower affinity AhR ligand, ³H-BNF. Again, EGCG failed to inhibit BNF binding to the AhR in Hepa cytosol (FIG. 2B). However, 3′-nitroflavone which has been demonstrated to weakly compete for binding with TCDD to the AhR and weakly inhibit TCDD-induced transcription very efficiently competed with BNF binding in this experimental system. This demonstrates that under these conditions low affinity ligands are capable of binding to the AhR ligand-binding site. Additional experimental adjustments to both treatment time and exposure temperature also failed to alter this experimental outcome for EGCG.

To further address the possibility that EGCG is a very low affinity AhR ligand, additional alterations in the experimental system were made that could possibly enhance its ability to compete. Denison et al. (J. Biol. Chem. 261:3987-3995 (1986), which is hereby incorporated by reference in its entirety) and Prokipcak et al. (Arch. Biochem. Biophys. 267:811-828 (1988), which is hereby incorporated by reference in its entirety) suggest that binding of ARNT to the TCDD-AhR complex results in increased stabilization of the TCDD-AhR interaction into a nearly irreversible complex. If this is occurring, a ligand with low binding affinity may not be able to effectively compete with ³H-TCDD under conditions that shift the equilibrium to favor a TCDD-AhR-ARNT complex. Therefore, ligand binding was re-assessed in ARNT deficient cytosol obtained from BP^{r}Cl cells. In addition, lower concentrations of ³H-TCDD were used to further favor competitive binding by a weak ligand. Despite these alterations, EGCG was incapable of displacing TCDD from the ligand-binding site. Pretreatment of BP^{r}Cl cytosol for 30 minutes with EGCG also failed to inhibit TCDD binding. Together, these data support a mechanism of action that does not involve direct binding of EGCG to the TCDD ligand-binding site on the AhR.

### Example 3

### Hsp90 and XAP2 are Eluted From EGCG-Conjugated Sepharose Beads

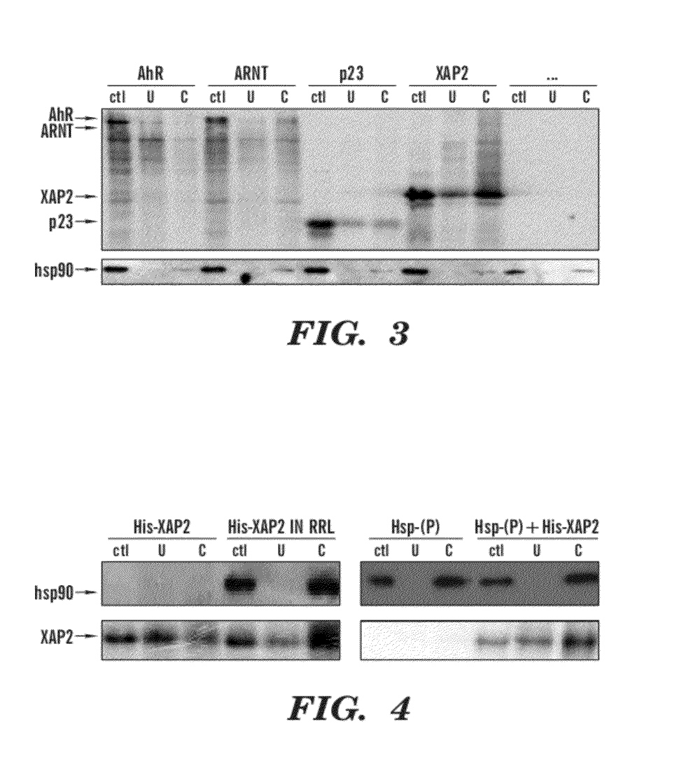

Based on the above competitive binding experiments, it is unlikely that EGCG is binding to the TCDD ligand-binding site on the AhR. This suggests that EGCG is either binding another site on the AhR or is affecting AhR activity through an indirect mechanism, perhaps involving binding to another protein in the AhR complex such as hsp90, XAP2, p23, or ARNT. To address these possibilities affinity chromatography was performed using EGCG-conjugated Sepharose. XAP2, ARNT, p23, and AhR proteins were separately transcribed in vitro in the presence of ³⁵S-methionine and incubated with either unconjugated Sepharose or EGCG-Sepharose. Binding of these proteins to the Sepharose beads was assessed by Phosphoimaging following SDS-PAGE of the eluted protein. Hsp90 is inherent to RRL, therefore the ability of this protein to bind EGCG was assessed by immunoblotting. As shown in FIG. 3, in vitro translated AhR was not able to bind immobilized EGCG. This supports the competitive binding data suggesting that EGCG does not bind the TCDD ligand binding site and also suggests that EGCG is not binding another site on the AhR. ARNT and p23 were also incapable of binding immobilized EGCG. However, both hsp90 and XAP2 were eluted from EGCG-Sepharose, implicating these two proteins as direct targets of EGCG. Hsp90 and XAP2 were also eluted from EGCG-Sepharose incubated with Hepa cytosol demonstrating that this interaction is not specific to this in vitro system.

### Example 4

### EGCG Binds an Hsp90-XAP2 Associated Complex

It has been demonstrated that XAP2 binds hsp90-AhR complexes and is also capable of binding both proteins independently (Meyer et al., Biochemistry 38:8907-8917 (1999), which is hereby incorporated by reference in its entirety). Therefore, it is conceivable that hsp90 and XAP2 are being eluted as a complex. Considering this, the data in FIG. 3 can be interpreted in two ways: 1) EGCG binds two different proteins in the AhR complex, or 2) EGCG binds one protein directly and the other indirectly through a protein-protein interaction. To delineate between these two possibilities, in vitro transcribed histidine-tagged XAP2 was purified using metal affinity chromatography. Purified XAP2 and purified hsp90 (commercially available) were incubated with EGCG-Sepharose alone, or in combination. The beads were washed and bound protein eluted. As shown in FIG. 4, purified hsp90 was eluted specifically from EGCG-Sepharose, strongly implicating a direct interaction between this ligand and the hsp90 protein. Interestingly, purified XAP2 was not specifically eluted from immobilized EGCG. Note that this purified protein is devoid of detectable hsp90 (FIG. 4, lane 1). However, reconstitution of His-purified XAP2 with purified hsp90 or RRL restores XAP2 elution, strongly suggesting that EGCG directly targets hsp90 and XAP2 is indirectly eluted as a result of an hsp90 interaction.

EGCG Binds the C-terminus of Hsp90. Hsp90 is composed of well-conserved amino- and carboxyl-terminal regions both containing ATP binding domains (Marcu et al., J. Biol. Chem. 275:37181-37186 (2000); Prodromou et al., Cell 90:65-75 (1997); Haystead et al., Eur. J. Biochem. 270:2421-2428 (2003), which are hereby incorporated by reference in their entirety). ATP binding and hydrolysis are essential for the activity of the protein (Prodromou et al., Cell 90:65-75 (1997), which is hereby incorporated by reference in its entirety) and inhibition of ATP binding to either domain has been demonstrated to disrupt the chaperone activity of hsp90 and therefore the activity of the client protein (Grenert et al., J. Biol. Chem. 272:23843-23850 (1997); Kazlauskas et al., Mol. Cell. Biol 21:2594-2607 (2001); Marcu et al., J. Natl. Cancer. Inst. 92:242-247 (2000); Yun et al., Biochemistry 43:8217-8229 (2004), which are hereby incorporated by reference in their entirety). However, inhibition of the N-terminal domain alters hsp90-complex maturation very differently than inhibition of the C-terminal domain, suggesting these two domains serve different functions. Furthermore, these domains possess distinct nucleotide binding specificity (Haystead et al., Eur. J. Biochem. 270:2421-2428 (2003), which is hereby incorporated by reference in its entirety) which provides a means for separating the functions of these domains with the use of site specific inhibitors (Marcu et al., J. Biol. Chem. 275:37181-37186 (2000), which is hereby incorporated by reference in its entirety). Therefore, identification of the EGCG binding site on hsp90 was considered important to understand its effects on hsp90 complex association.

To determine which site was responsible for the interaction between EGCG and hsp90, several hsp90 mutants were tested for their ability to bind to immobilized EGCG. Geldanamycin (“GA”) is known to specifically bind the N-terminus of hsp90 (Grenert et al., J. Biol. Chem. 272:23843-23850 (1997), which is hereby incorporated by reference in its entirety). As shown in FIG. 5, the amino-terminal hsp90 fragment containing the GA binding site (C507) failed to bind to immobilized EGCG. Furthermore, several N-terminal point mutants (D92A, G94D) known to abrogate GA binding to hsp90 (Grenert et al., J. Biol. Chem. 272:23843-23850 (1997), which is hereby incorporated by reference in its entirety) bound to EGCG-Sepharose as well as or better than the wild type hsp90. Interestingly, all C-terminal fragments bound EGCG. Analysis of the smallest truncation mutant containing amino acids 538-728 (N538) revealed that binding of EGCG occurs within this region of the protein, suggesting an interaction with the C-terminal region near or at the ATP binding site (FIG. 5).

### Example 5

### EGCG Induced Nuclear Localization of the AhR

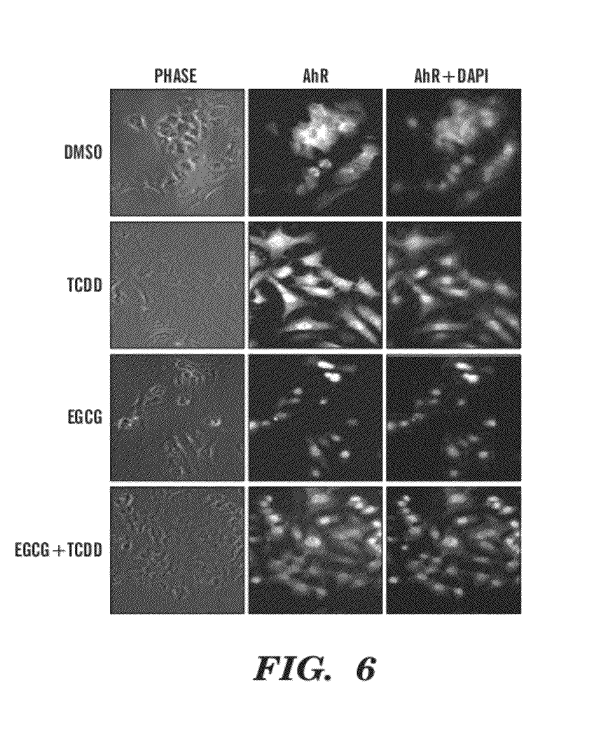

To begin to understand the molecular consequences of the EGCG-hsp90 interaction on AhR function it was important to determine which processes within the AhR activation pathway EGCG is capable of inhibiting. After ligand binding, the next well-defined event required for AhR gene activation to occur is nuclear localization. To assess the effects of EGCG treatment on nuclear uptake of the AhR, Hepa cells were treated and the subcellular localization of the AhR visualized by immuno fluorescence microscopy. As expected, treatment of cells with TCDD for one hour resulted in a redistribution of the AhR from the cytosol to the nucleus (FIG. 6) (Pollenz et al., Mol. Pharmacol. 45:428-38 (1994), which is hereby incorporated by reference in its entirety). Treatment with EGCG did not attenuate this TCDD induced nuclear localization of the AhR. Interestingly, when cells were exposed to EGCG alone there was a remarkable redistribution of the AhR from the cytosol to the nucleus. In fact, EGCG treatment alone was just as good if not better at nuclear redistribution of the AhR than TCDD. These data indicate that binding of EGCG to hsp90 substantially alters the conformation or protein-protein interactions of the AhR complex, resulting in a redistribution of the AhR to the nuclear compartment of the cell. These observations suggest that EGCG affects the AhR within the cytoplasmic compartment of the cell. These data also demonstrate that nuclear localization of the AhR does not necessarily reflect transcriptional activity of this protein.

### Example 6

### EGCG Inhibits TCDD Induced Binding of the AhR to Dioxin Responsive Elements

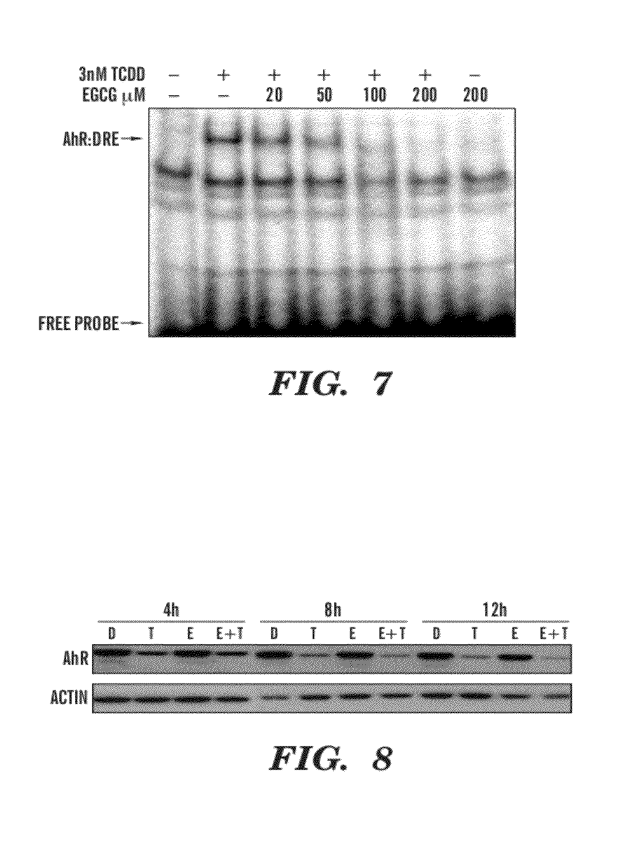

Considering that the EGCG-bound AhR was localized to the nucleus, it was important to determine if EGCG was inhibiting TCDD-mediated gene induction through a mechanism involving inhibition of the AhR-DNA interaction. To address this question, Hepa cytosol was incubated with EGCG in the presence and absence of TCDD and DNA binding forms analyzed by EMSA. Cytosol treated with EGCG showed a concentration dependent decrease in the TCDD-AhR-DRE shifted band (FIG. 7). This compound was able to inhibit binding by nearly 100% at 100 μM, providing evidence that EGCG alters the ability of a TCDD-bound AhR to transform into a DNA binding form. EGCG had the same effect on TCDD-induced binding when extracts from treated whole cells were isolated. In both systems, DRE binding was not observed with EGCG treatment alone.

EGCG does not affect AhR degradation. It is well established that the AhR protein is downregulated under many experimental conditions following agonist exposure both in vivo and in whole cells (Pollenz, Chem. Res. Toxicol. 141:41-61 (2002), which is hereby incorporated by reference in its entirety). Specifically, in Hepa cells the concentration of the AhR rapidly declines after 2 h of TCDD exposure (Pollenz et al., Mol. Pharmacol. 45:428-38 (1994); Pollenz, Mol. Pharmacol. 49:391-398 (1996), which are hereby incorporated by reference in their entirety), resulting in a dramatic decrease in the half-life of the AhR (Ma et al., J. Biol. Chem. 275:8432-8438 (2000), which is hereby incorporated by reference in its entirety). Previous reports have demonstrated that inhibition of this ligand induced degradation results in an increase in the magnitude and duration of the induction of AhR-responsive genes (Ma et al., J. Biol. Chem. 275:8432-8438 (2000); Ma et al., J. Biol. Chem. 275:12676-12683 (2000), which are hereby incorporated by reference in their entirety). Conversely, it has been demonstrated that TCDD induced gene induction can be reduced as a result of AhR degradation (Song et al., Mol. Pharmacol. 62:806-816 (2002), which is hereby incorporated by reference in its entirety). These data emphasize the importance of AhR protein concentration in the response of cells to ligands, indicating that downregulation of the AhR serves a role in the attenuation of the gene regulatory response.

The importance of AhR stability and its effect on gene induction have been further emphasized in studies utilizing the hsp90 inhibitor GA. Exposure to GA inhibits TCDD-induced gene transcription through a mechanism involving destabilization of the hsp90-AhR complex resulting in rapid proteolysis of the AhR (Song et al., Mol. Pharmacol. 62:806-816 (2002); Chen et al., Arch. Biochem. Biophys. 348:190-198 (1997), which are hereby incorporated by reference in their entirety). Based on this knowledge it is possible that EGCG could be inhibiting AhR gene induction through a mechanism involving increased protein degradation. To assess this possibility, cells were treated with either DMSO, TCDD, EGCG, or EGCG in the presence of TCDD over a time period of 12 hours and the levels of AhR protein determined by western blotting. As expected, AhR levels decline rapidly upon exposure to TCDD (FIG. 8). This decrease was not exacerbated or prevented by simultaneous treatment with EGCG. Furthermore, treatment with EGCG alone was not capable of inducing AhR degradation, supporting the hypothesis that EGCG does not destabilize the AhR-hsp90 interaction.

### Example 7

### EGCG Affects AhR Complex Association Differently than TCDD

Sucrose density gradient centrifugation has been used extensively in the determination of molecular weights for individual and multiprotein complexes and to assess alterations in protein-protein interactions based on sedimentation coefficients within the gradient. The AhR in cytosolic samples sediments under conditions of low ionic strength as a specific peak in the ˜9.8S region of the sucrose density gradient (Okey et al., J. Biol. Chem. 254:11636-11648 (1979); Prokipcak et al., Arch. Biochem. Biophys. 267:811-828 (1988), which are hereby incorporated by reference in their entirety). Previous studies have established that this peak represents an AhR complex associated with two molecules of hsp90 (Wilhelmsson et al., EMBO J. 9:69-76 (1990); Probst et al., Mol. Pharmacol. 44:511-518 (1993), which are hereby incorporated by reference in their entirety). Interestingly, the sedimentation properties of the cytosolic AhR that has been transformed to a DNA-binding form in vitro have been reported to be the same as those of the native cytosolic AhR (Hannah et al., Eur. J. Biochem. 156:237-242 (1986), which is hereby incorporated by reference in its entirety). This is observed in the sucrose density gradient experimental system and is demonstrated in FIG. 2 where incubation of Hepa cytosol with ³H-TCDD resulted in a specific peak of radioactivity approximately spanning fractions 8-14 (˜9S fraction).

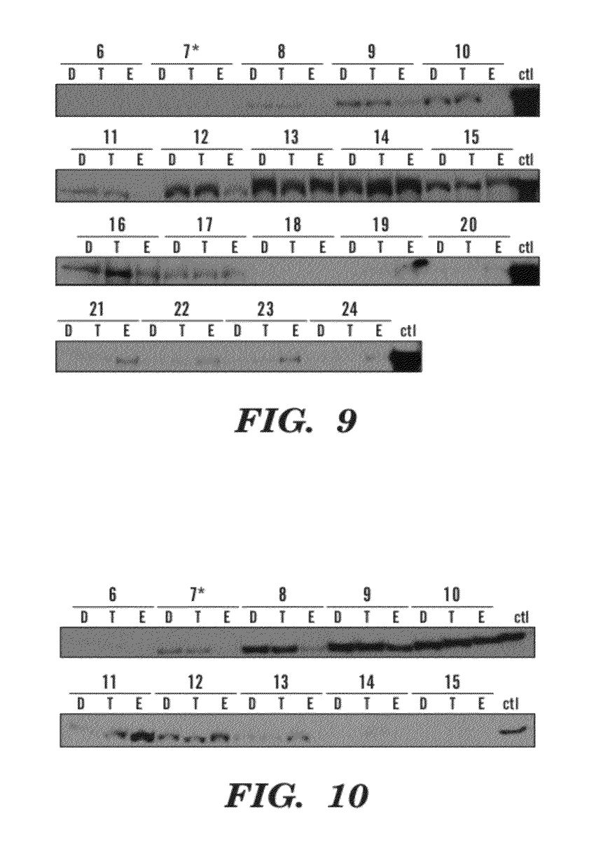

Sucrose density gradients were used to determine the effects of EGCG on the sedimentation properties of the AhR. These experiments demonstrate that upon exposure of Hepa cytosol to TCDD, the AhR sediments in fractions 8-16 (FIG. 9). This sedimentation profile corresponds to the ˜9S region and correlates with the radioactive peak observed in FIG. 2 upon detection of bound ³H-TCDD. This sedimentation pattern is similar to the sedimentation pattern in the absence of ligand with the exception of a slightly higher density shift associated with TCDD treatment. This higher level of AhR in fraction 12 upon exposure to TCDD was consistently observed in repeat experiments. Interestingly, EGCG exposure consistently resulted in a different sedimentation pattern. Within the 9S region of the gradient, the EGCG-treated AhR sedimented predominantly in fractions 13-15. Furthermore, the AhR was detected within fractions 21-23 (˜13-14S) following EGCG treatment. These data suggest that EGCG affects the AhR-complex very differently than TCDD, strongly implicating altered conformation or protein-protein interactions in mediating the effects of EGCG on the AhR.

Considering EGCG targets hsp90 directly, it was of interest to assess its effects on hsp90 sedimentation as well. As shown in FIG. 10, hsp90 sediments predominantly within fractions 8-10 in an untreated cell. TCDD treatment did not appear to influence this sedimentation pattern. However, EGCG treatment dramatically altered the sedimentation pattern of hsp90 as demonstrated by the loss of hsp90 detection in fractions 7-8 and an increase in hsp90 detection in fraction 11-13. Although the limitations of this system prevent us from drawing any conclusions regarding hsp90-AhR association, these experiments strongly suggest that EGCG affects hsp90 and that these effects are not specific to the hsp90 bound to the AhR.

### Discussion of Examples 1-7

These experiments demonstrate that EGCG, a compound with structural similarity to known AhR ligands, inhibits AhR activity through a mechanism that does not involve direct binding to the TCDD ligand binding site. To date, most, if not all, AhR antagonists have been found to bind to the AhR protein. The present data strongly emphasize that competing for binding to the AhR ligand binding site is not the only mechanism of action for AhR antagonists and that structural similarity to known AhR ligands does not necessarily imply competitive binding.

To conclusively determine that EGCG was not binding to the AhR it was important to adjust the experimental system to favor low affinity competition. Some of these modifications included: 1) decreasing the concentration of TCDD, 2) increasing the concentration of EGCG, and 3) using the lower affinity competing ligand, BNF. Successful competition by the low affinity ligand 3′-nitroflavone indicated that these conditions were conducive for low affinity binding (FIG. 2B). However, EGCG was incapable of competing for binding to the AhR under any of these experimental conditions. Furthermore, in vitro translated AhR failed to bind EGCG-Sepharose (FIG. 3). Affinity chromatography using purified proteins further indicated that EGCG binds the C-terminus of hsp90 (FIG. 5) and indirectly elutes XAP2 as a result of the hsp90-XAP2 interaction (FIG. 4). Together, these data strongly imply that EGCG does not bind the ligand binding domain or any other domain on the AhR, and alters AhR activity through an interaction with hsp90.

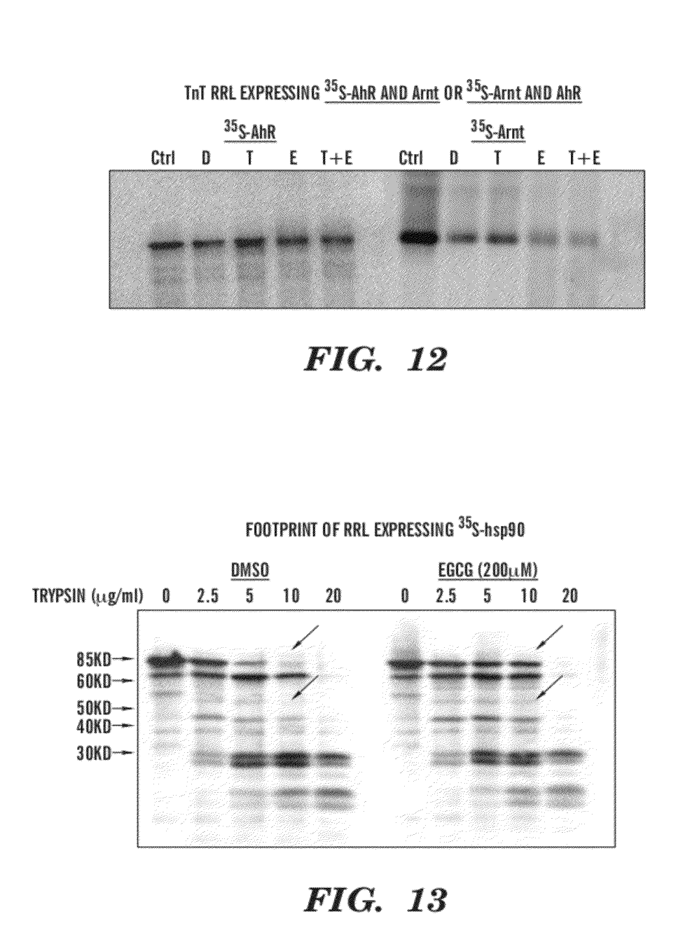

The observation that EGCG treatment alone induces a rapid and profound redistribution of the AhR to the nuclear compartment of the cell (FIG. 6) suggests that EGCG exerts its initial effect on the AhR within the cytoplasm. These data emphasize that EGCG is not necessarily functioning only to block a TCDD-induced effect, but is capable of modulating the receptor conformation/complex association on its own. Once in the nucleus, the EGCG-bound AhR complex is incapable of binding DREs (FIG. 7). Considering that the AhR cannot bind DNA in the absence of ARNT (Probst et al., Mol. Pharmacol. 44:511-518 (1993); Hoffman et al., Science 252:954-958 (1991), which are hereby incorporated by reference in its entirety), these data might suggest that EGCG inhibits ARNT dimerization. Although ARNT is necessary for DNA binding, recent data suggests it is not sufficient and DNA binding may require additional processes including phosphorylation (Minsavage et al., J. Biol. Chem. 279:20582-20593 (2004), which is hereby incorporated by reference in its entirety) or cofactor recruitment (Nguyen et al., Arch. Biochem. Biophys. 367:250-257 (1999), which is hereby incorporated by reference in its entirety). Therefore it is possible that EGCG binding results in Ahr-ARNT heterodimerization, yet inhibits the ligand induced conformational change necessary for these additional events. However, data presented in FIG. 12 strongly suggest that EGCG inhibits the interaction of AhR with ARNT.

In the cytoplasm, the AhR exists complexed with two molecules of hsp90.

These hsp90 molecules contact the AhR in two regions—the bHLH region located at the N-terminus of the protein and the PAS domain. Within the PAS domain, the AhR-hsp90 interaction overlaps with the ligand binding domain and the ARNT dimerization domain (Coumailleau et al., J. Biol. Chem. 270:25291-25300 (1995); Perdew et al., Biochem. Mol. Int. 39:589-593 (1996); Whitelaw et al., EMBO J. 12:4169-4179 (1993); Dolwick et al., Proc. Natl. Acad. Sci. 90:8566-8570 (1993), which are hereby incorporated by reference in their entirety), whereas within the basic region, it overlaps with both the DNA binding region (Dolwick et al., Proc. Natl. Acad. Sci. 90:8566-8570 (1993); Pongratz et al., Mol. Cell. Biol. 18:4079-4088 (1998), which are hereby incorporated by reference in their entirety) and the nuclear localization sequence (Eguchi et al., J. Biol. Chem. 272:17640-17647 (1997), which is hereby incorporated by reference in its entirety). These interactions result in an AhR conformation capable of binding ligand with high affinity (Carver et al., J. Biol. Chem. 269:30109-30112 (1994); Whitelaw et al., Proc. Natl. Acad. Sci. 92:4437-4441 (1995); Coumailleau et al., J. Biol. Chem. 270:25291-25300 (1995); Pongratz et al., J. Biol. Chem. 267:13728-13734 (1992), which are hereby incorporated by reference in their entirety) and incapable of ARNT dimerization due to steric interference (Coumailleau et al., J. Biol. Chem. 270:25291-25300 (1995); Perdew et al., Biochem. Mol. Int. 39:589-593 (1996); Whitelaw et al., Mol. Cell. Biol 13:2504-2514 (1993), which are hereby incorporated by reference in their entirety). In response to ligand, two possible pathways for the AhR have been proposed: 1) the AhR complex dissociates in the cytoplasm and free AhR becomes associated with nuclear transport proteins to be translocated to the nucleus, or 2) ligand binding initiates nuclear translocation of the intact complex where hsp90 and XAP2 dissociate prior to, or in concert with, dimerization with ARNT. The data shown here are consistent with a model in which EGCG binding to hsp90 results in a conformational change responsible for a modification of the hsp90-AhR interaction with the bHLH region of the AhR and increased stabilization of the PAS-hsp90 interaction. This results in exposure of the nuclear localization signal, supporting a model for nuclear localization of an hsp90-associated AhR complex. In the nucleus, increased stabilization of the hsp90-AhR interaction within the PAS domain prevents further dissociation of this complex and possibly ARNT dimerization. Ligand-induced translocation of the AhR-hsp90 core complex prior to dissociation has been previously suggested supporting the model proposed here (Wilhelmsson et al., EMBO J. 9:69-76 (1990); Heid et al., Mol. Pharmacol. 57:82-92 (2000); Petrulis et al., J. Biol. Chem. 275:27448-27453 (2000), which are hereby incorporated by reference in their entirety).

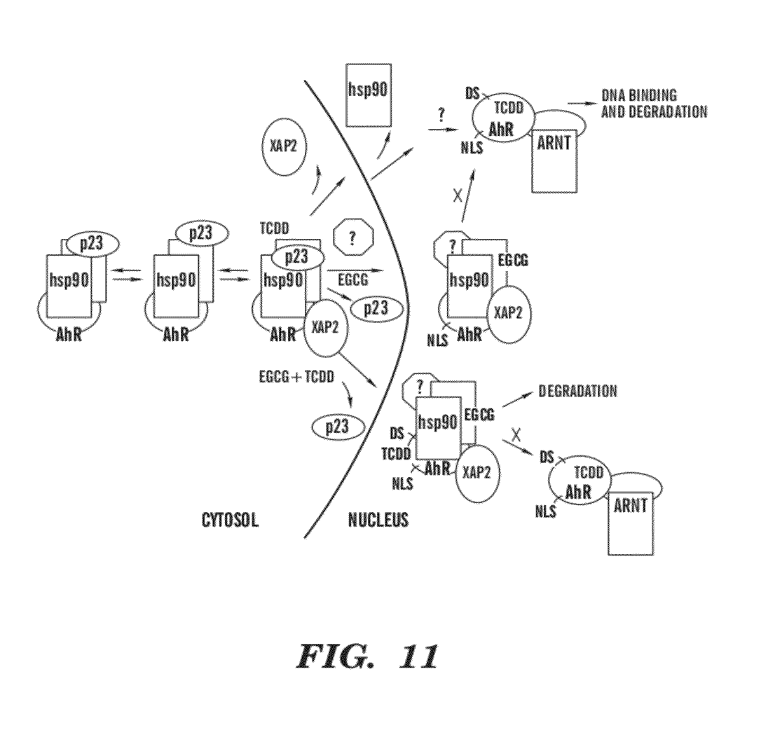

The above results suggest a model in which EGCG maintains AhR protein levels through stabilization of the AhR-hsp90 association. However, if this is occurring, then why upon simultaneous treatment with TCDD does the AhR still undergo TCDD mediated degradation (FIG. 8)? It is well established that the AhR-hsp90 interaction is an important determinant of AhR stability (Song et al., Mol. Pharmacol. 62:806-816 (2002); Heid et al., Mol. Pharmacol. 57:82-92 (2000), which are hereby incorporated by reference in their entirety). However, the processes responsible for mediating ligand-induced degradation of the receptor remain unclear. It is proposed that there are two distinct signals for AhR degradation—one mediated by the dissociation and/or altered binding of hsp90 and its associated proteins, and the other by the agonist-elicited activation of the AhR. Currently, GA-mediated degradation of hsp90 client proteins, including the AhR, is thought to occur through a CHIP— (C-terminal hsp70-interacting protein) mediated mechanism. Although the details that control these pathways are poorly understood, CHIP initiates degradation through a process involving binding to the tetratricopeptide repeat motif (TPR) on the hsp90/hsp70 chaperone complex, ubiquitination, and the 26S proteasome (Connell et al., Nature Cell Biol. 3:93-96 (2001); Ballinger et al., Mol. Cell. Biol 19:4535-4545 (1999); Jiang et al., J. Biol. Chem. 276:42938-42944 (2001), which are hereby incorporated by reference in their entirety). Although GA induces degradation of the AhR to the same degree as TCDD (Song et al., Mol. Pharmacol. 63:597-606 (2003), which is hereby incorporated by reference in its entirety), it should be noted that other data suggests that these two degradation processes are presumably distinct. Song and colleagues demonstrated that GA-mediated degradation occurs at a much faster rate than TCDD-mediated degradation (Song et al., Mol. Pharmacol. 63:597-606 (2003), which is hereby incorporated by reference in its entirety). GA-mediated degradation is not altered by the nuclear export inhibitor leptomycin B, whereas TCDD-mediated degradation is (Song et al., Mol. Pharmacol. 62:806-816 (2002), which is hereby incorporated by reference in its entirety). Furthermore, TCDD induces an AhR conformation void of its hsp90 chaperone complex. By releasing this complex, the TCDD-activated AhR loses its link to the CHIP-mediated degradation pathway necessitating an alternative signal in the TCDD-mediated degradation pathway. In the model proposed here (FIG. 11), stabilization of the AhR-hsp90 complex by EGCG may only affect the stronger interactions with the PAS domain of the AhR and may not interfere with the potential ligand-induced changes necessary for degradation. Alone, EGCG may maintain the hsp90 conformation in a state incapable of recruiting degradation cofactors such as CHIP. However, when TCDD binds in the presence of EGCG, the AhR conformation is altered exposing the AhR degradation signal resulting in ligand induced degradation following nuclear localization. Interestingly, a TPR half site has been identified (Levine et al., Mol. Pharmacol. 58:1517-1524 (2000), which is hereby incorporated by reference in its entirety) in the N-terminal region of the AhR and it may be that CHIP, or another unknown protein, binds directly to this site to mediate degradation. It has also been demonstrated that deletion of the transactivation domain of the AhR greatly reduces TCDD-mediated degradation (Ma et al., J. Biol. Chem. 275:8432-8438 (2000), which is hereby incorporated by reference in its entirety), suggesting the degradation signal may be contained within the C-terminal domain of the receptor. In any case, it is proposed that TCDD binding to the AhR results in a conformational change responsible for exposing a proposed degradation signal on the AhR that is not dependent on its association with hsp90. These observations strongly implicate the involvement of distinct protein-protein interactions, and/or AhR domains in the degradation of the inactive versus the TCDD-activated AhR complex.

The data provided above implicate a model where EGCG inhibits release of hsp90 from the AhR complex. This form of hsp90 appears to maintain its interaction with XAP2 (FIG. 4). However, from the above data, it remains to be determined whether 1) the hsp90-XAP2 complex remains bound to the AhR upon EGCG treatment, 2) EGCG prevents ARNT dimerization, 3) EGCG stabilizes the receptor in a different complex upon additional treatment with TCDD, and 4) if other proteins are present in this complex. Findings from the above investigations add some useful insight into the mechanism of action of EGCG. The mouse AhR used for these studies was in vitro transcribed in rabbit reticulocyte lysate (RRL) and immunoprecipitated using an antibody specific for amino acids 12-31. Although this antibody could precipitate the latent and TCDD-activated AhR complex from RRL, it was incapable of precipitating an EGCG-treated AhR. Immunoprecipitation under denaturing conditions restored the ability to immunoprecipitate the AhR following EGCG treatment, suggesting that an EGCG-induced conformational change was responsible for the lack of antibody recognition. Interestingly, this conformational change did not appear to occur in Hepa cytosol because the AhR could be successfully immunoprecipitated from this system following EGCG treatment. Furthermore, titration of Hepa cytosol into the RRL system restored the ability to immunoprecipitate ³⁵S-methionine-labeled in vitro transcribed AhR. Similar discrepancies between systems were observed utilizing DNA binding as an endpoint. EGCG treatment of AhR and ARNT in RRL resulted in the formation of a strong DRE-AhR-ARNT shifted complex. This is in direct contrast to the lack of AhR transformation by EGCG observed in Hepa cytosol (FIG. 7). Furthermore, the EGCG-induced DNA binding complex had a different mobility than the TCDD-induced DNA binding complex, suggesting altered conformation or protein association following AhR activation by EGCG as compared to TCDD in the RRL system. This EGCG-induced DNA binding complex could also be attenuated upon titration of Hepa cytosol into RRL. Together these observations support the presence of an additional inhibitory factor within Hepa cytosol that is not present in RRL and that is pertinent to the antagonist affects of EGCG on the AhR complex.

Comparison of the effects of EGCG treatment on the AhR signaling pathway with those of two other characterized hsp90 inhibitors suggests a unique mechanism of inhibition. Binding of GA to the N-terminal ATP binding pocket on hsp90 functions to destabilize the hsp90-AhR interaction resulting in release of p23 and XAP2 (Kazlauskas et al., J. Biol. Chem. 274:13519-13524 (1999); Kazlauskas et al., Mol. Cell. Biol 21:2594-2607 (2001); Kazlauskas et al., J. Biol. Chem. 275:41317-41324 (2000); Sullivan et al., J. Biol. Chem. 272:8007-8012 (1997), which are hereby incorporated by reference in their entirety), signaling the AhR for degradation (Song et al., Mol. Pharmacol. 62:806-816; Chen et al., Arch. Biochem. Biophys. 348:190-198 (1997), which are hereby incorporated by reference in their entirety). Conversely, the inhibitor molybdate stabilizes the hsp90-AhR interaction through an unknown mechanism, resulting in enhanced association with p23 and stabilization of the AhR protein (Kazlauskas et al., J. Biol. Chem. 274:13519-13524 (1999); Heid et al., Mol. Pharmacol. 57:82-92 (2000), which are hereby incorporated by reference in their entirety). Interestingly, EGCG appears to alter AhR function in an intermediate manner. Based on the data in FIG. 4 suggesting that EGCG targets an hsp90-XAP2 complex, it is proposed that EGCG retains the AhR in a conformation that remains bound to both hsp90 and XAP2. This retention of XAP2 directly reflects a particular conformational state of hsp90 and its presence within the AhR complex has important functional consequences. In the literature, XAP2 has been implicated in many processes including enhancing AhR stability (Meyer et al., Biochemistry 38:8907-8917 (1999); Kazlauskas et al., J. Biol. Chem. 275:41317-41324 (2000), which are hereby incorporated by reference in their entirety), decreasing AhR ubiquitination (Kazlauskas et al., J. Biol. Chem. 275:41317-41324 (2000), which is hereby incorporated by reference in its entirety), and enhancing nuclear targeting (Ma et al., J. Biol. Chem. 272:8878-8884 (1997), which is hereby incorporated by reference in its entirety). In the model proposed here, the simultaneous contact of XAP2 with the AhR and hsp90 in the presence of EGCG, may enhance the AhR-hsp90 interaction resulting in increased AhR stability and protein levels and increase nuclear uptake. More importantly, XAP2-mediated enhancement of the hsp90-AhR interaction may be pertinent in preventing the AhR from binding DNA.

Currently, there is extensive literature addressing the numerous biological effects of EGCG on cellular function including inhibition of telomerase (Naasani et al., Biochem. Biophys. Res. Commun. 249:391-396 (1998), which is hereby incorporated by reference in its entirety) and kinase (Yang et al., Mol. Pharmacol. 60:528-533 (2001); Chung et al., FASEB J. 15:2022-2024 (2001); Liang et al., J. Cell Biochem. 75:1-12 (1999), which are hereby incorporated by reference in their entirety) activities, as well as altering the normal function of numerous transcription factors (Yang et al., Mol. Pharmacol. 60:528-533 (2001); Ren et al., Oncogene 19:1924-1932 (2000); Kuruto-Niwa et al., J. Agric. Food Chem. 48:6355-6361 (2000), which are hereby incorporated by reference in their entirety). Interestingly, a large number of these affected proteins are also hsp90-client proteins. The observed shift in the sedimentation rate of hsp90 following EGCG treatment (FIG. 10) suggests that EGCG may target any and all cellular hsp90. This overlap between hsp90 inhibition by EGCG and EGCG inhibition on numerous hsp90-client proteins provides a very desirable explanation for how one compound, and green tea, could have so many biological effects.

EGCG inhibits AhR transcriptional activation through an indirect mechanism involving direct binding of EGCG to the C-terminus of the AhR chaperone protein hsp90. This is the first time EGCG has been demonstrated to bind hsp90 directly and therefore the first indication that this compound may function as an hsp90 inhibitor. EGCG appears to target a XAP2-bound hsp90 complex suggesting this compound inhibits hsp90 function differently than currently reported hsp90 inhibitors. Elucidation of the effects of hsp90 inhibition by EGCG on AhR stability, DNA binding activity, cellular localization and protein-protein interactions will help to refute or support a unique mechanism of hsp90 inhibition and provide further insight regarding how AhR associated proteins are involved in receptor regulation.

### Example 8

### EGCG Affects AhR/Arnt Complex Association

AhR and Arnt were separately translated in vitro using the TNT RRL system as described supra. For each experiment, only one of the proteins translated in the presence of [³⁵S]Methionine. Equal volumes of diluted AhR and Arnt translation were mixed, incubated with DMSO, 1 nM TCDD, 200 μm EGCG, or TCDD plus EGCG and immunoprecipitated with anti-AhR antibody. All samples were separated by 7.5% SDS-PAGE, transferred to PVDF membrane, and visualized by phosphorImager. Results of the experiment are illustrated in FIG. 12. FIG. 12 demonsrates that EGCG affects AhR's ability to interact with Arnt, thereby disrupting AhR/Arnt complex formation.

### Example 9

### EGCG Binding to Hsp90 Alters Hsp90 Conformation

Chicken hsp90 was translated in vitro using the TNT RRL system as described supra in the presence of [³⁵S]Methionine, diluted, incubated with DMSO or EGCG, and treated with trypsin at indicated concentrations for 10 minutes at room temperature. All samples were separated by 10% SDS-PAGE, transferred to PVDF membrane, and visualized by phosphorImager. Results of the experiment are illustrated in FIG. 13. FIG. 13 demonstrates direct binding of EGCG to hsp90, resulting in an altered conformation of hsp90. Notably, this experiment was performed in the absence of AhR or ARNT. Therefore, the ability of EGCG to affect hsp90 conformation is not dependent on the presence of AhR or ARNT.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method of treating a cancerous condition comprising:
inhibiting an interaction between hsp90 and a protein encoded by a gene selected from the group consisting of pS2, cathepsin D, Sp1, heat shock protein 27, T cadherin, latent transforming growth factor-β binding protein 1, aryl hydrocarbon receptor repressor (AhRR), NAD(P)H-menadione oxidoreductase 1, plasminogen activator inhibitor-2, ecto-ATPase, interleukin-2, cyclooxygenase-2, UDP glucuronosyltransferase 1, glutathione-S-transferase Ya, CYP1A1, plasminogen activator inhibitor-1, CYP1B1, aldehyde dehydrogenase 4, hairy and enhancer of Split homolog-1 (HES-1), CYP1A2, paraoxonase, proopiomelanocortin (ACTH precursor), c-myc, transforming growth factor-beta, interleukin-6, interferon-gamma, poly(ADP-ribose) polymerase, BSAP, Bax, polκ, DIF-3, Cu/Zn superoxide dismutase, CYP2S1, steroidogenic acute regulatory protein, RANTES, MHC Q1, transforming growth factor-alpha (TGF-α), urokinase plasminogen activator, Interleukin-1β, c-fos, c-jun, ADP ribosylation factor 4, basic transcription factor 2 (34-kDa subunit), cadherin 2, CDC-like kinase, complement component 5, cyclin-dependent kinase inhibitor 1A, cyclin-dependent kinase 1, CYP19A1, DNA mismatch repair protein, early growth response protein, 110-kDa heat-shock protein, heat shock factor-binding protein 1, 60-kDa heat shock protein, insulin-like growth factor-binding protein 10, insulin-like growth factor binding protein 1, insulin-like growth factor II, integrin β, interleukin 1 receptor type 1, 45-kDa interleukin enhancer-binding factor 2, NEDD5 protein homolog, Niemann-Pick C disease protein, retinoblastoma-binding protein 3, Rab geranylgeranyl transferase β subunit, RNA polymerase II elongation factor SIII p15 subunit, Sec61-γ; sex-determining region Y box-containing gene 9, short/branched chain-specific acyl-CoA dehydrogenase, solute carrier family 2 member 2, T-complex protein 1 τ and δ subunits, thyroid receptor-interacting protein 15, topoisomerase I and II α, transcription factor HTF4, translation initiation factor 4E 25-kDa subunit, CYP2C11, albumin, ATP synthetase β subunit, calreticulin precursor, cytochrome B5, CYP2D4, 25DX, endoplasmic reticulum protein ERP29 precursor, ferritin light chain, 78 kDa glucose-regulated protein precursor, glutamate dehydrogenase, glyceraldehydes-3-phosphate dehydrogenase, heat shock protein 72, 3-α-hydroxysteroid dehydrogenase, IκB kinase 2, 150 kDa iodothyronine 5′ monodeiodinase, isocitrate dehydrogenase, oxygen-regulated protein, peroxiredoxin IV, prohibitin, protein disulfide isomerase ER60 precursor, Bcl-2 family genes (bik, bid, Hrk, bok/mtd, mcl-1, bcl-x, and bcl-w), IAP family genes (X-linked IAP, NAIP1, and NAIP5), Myd88, p21, p53, RIP, TNFR, family genes (OX40, Fas, CD30, Ltβ-R, and TNFR1), TNF family genes (LIGHT, OX40L, and Bar-like), TRAF2, lecithin:retinol acyltransferase, actin α, Ahr, alcohol dehydrogenase 1 complex, angiopoietin-like 4, angiotensinogen, brain derived neurotrophic factor, cadherin 16, calbindin-28k, carbonic anhydrase 3, carboxylesterase 3, Cd44 antigen, coagulation factor II, cytokine receptor-like factor 1, epiregulin, fibroblast growth factor 7, fibroblast growth factor receptor 4, follistatin, forkhead box a2 and f2, Fos-like antigen 1, glutamyl aminopeptidase, Gro1 oncogene, high mobility group at-hook 2, α-2-hs-glycoprotein, hydroxysteroid 11-β dehydrogenase 2, insulin-like growth factor 2, insulin-like growth factor binding proteins 3, 5, and 6, integrin α 3, α 6 and β 4, IL-6, interferon activated gene 202a, lymphocyte antigen 6 complex (loci e, A and H), lysyl oxidase, matrix metalloproteinase 3 and 9, mitogen regulated protein proliferin 3, NADH dehydrogenase 1, osteopontin, p21, peripherin, phospholipase a2 group VII, proliferin 2, Ras-related protein, rennin 1 structural, retinol binding protein 4, plasma, RNA binding motif, single stranded interacting protein 1, secreted phosphoprotein 1, small proline-rich proteins 2b, 2c and 2f, spleen tyrosine kinase, squalene epoxidase, stratifin, thrombomodulin, TNF receptor family member 1b, tumor-associated calcium signal transducer 2, ADP-ribosylation-like factor 6 interacting protein 5, calcium binding protein All, CCAAT/enhancer-binding protein, esterase 10, immediate early response 3, nicotinic acetylcholine receptor subunit α 6, nuclear factor erythroid derived 2, like 2, prenylated SNARE protein, RIKEN-CDNA FLJ13933 FIS, clone Y79AA1000782, RIKEN-phosphogluconate dehydrogenase inhibitor, 5100 calcium-binding protein A4, vanin 1, Vomeronasal organ family 2, receptor 11, distal-less homeobox 5, activin receptor type II B, acyl-coenzyme A oxidase, aminoacylase 1, B-cell lymphoma protein 3, basic transcription element binding protein 1, bone morphogenic protein, β-catenin, Cdc42, CDK-2 associated protein, cellular retinoic acid binding protein 1, collagen IV α 3 chain, collagen VI α 3, cyclin-dependent kinase 4 inhibitor C, cyclin-dependent kinase inhibitor 2B iso form, CYP27A1, discoidin receptor tyrosine kinase, E2F dimerization partner 2, early growth response 1, EGF-containing fibulin-like extracellular matrix protein, ephrin A1 (isoform a), epidermal growth factor receptor substrate 15, epithelial-cadherin, fibroblast growth factor, fibronectin receptor β subunit, fos-related protein, GABA A receptor, GATA binding protein 1, glucocorticoid receptor, GTPase activating protein, homospermidine synthase, hsp 70 kDa protein insulin-like growth factor 1 receptor, GABA A receptor ε subunit, 25 kDa GTP binding protein, 1 hsp 70 kDa 2, hyaluronidase 1, insulin induced protein 1, interferon-induced protein 56 and p78, interferon γ receptor 1, interferon regulatory factor 4, IL-6 receptor β, IL-8, Kruppel-like factor 5, lamanin B2 chain and α 3b chain, leukemia inhibitor factor, low density lipoprotein receptor-related protein, macrophage inflammatory protein 1-β, MAP kinase-activated protein kinase 2, MAP kinase phosphatase-1, matrix metalloproteinase 1 and 9, mesoderm specific transcript iso form, mitotic arrest defective protein, multifunctional DNA repair enzyme, neurotrophic tyrosine kinase, NFκB p100/p49 subunits, nuclear receptor coactivator 2, ornithine cyclodeaminase, 8-oxo-dGTPase, p53, p53-binding protein Mdm4, peripheral benzodiazepine receptor, polyamine oxidase, protein kinase C α, protein kinase C-like 2, protein tyrosine phosphatase type 1, pyruvate dehydrogenase kinase, replication licensing factor, retinoic acid receptor β, RNA polymerase II, S100 calcium binding protein, serine/threonine kinase 4, serine/threonine specific protein phosphatase, serum/glucocorticoid regulated kinase, STAT1, thioltransferase, thioredoxin reductase, thrombin receptor, thrombomodulin, thymosin β 10, tissue inhibitor of metalloproteinase-3, translation initiation factor 3 and 4H, transmembrane 4 superfamily member, tumor-associated calcium signal transducer 4, tyrosine-protein kinase receptor, ubiquitin-like interferon, α-inducible protein, vasoactive intestinal polypeptide receptor, VEGF, vitronectin, WAP four-disulfide core domain 2 (isoform 1) precursor, zinc finger protein 42, DEAD/H box polypeptide 3, DnaJ (hsp40) homolog (subfamily B, member 1), fatty acid binding protein 2 (intestinal), heat shock 70 kDa protein 5, heat shock protein 1a (hsp90), heat shock protein 105, hepatic nuclear factor 4 (HNF4), HIV-tat interactive protein 2, homocysteine-inducible ER stress-inducible ubiquitin-like domain member 1 (Herp), C-type lectin-like receptor 2, lectin (galactose binding, soluble 1), malic enzyme, mannoside acetylglucosaminyltransferase 2, phosphoribosyl pyrophosphate amidotransferase, pleckstrin homology domain containing (family B number 1), Ras homolog gene family member E, ribosomal protein L12, S-100 calcium binding protein A10 (calpactin), signal transducer and activator of transcription 2, solute carrier protein 21 (organic anion transporter, member 10), TNFα-induced adipose-related protein, ubiquitin-specific protease 2, vaccinia related kinase 2, zinc finger protein 191, matrix metalloproteinase-1, CK8 polypeptide, glutathione peroxidase, Ig lambda-1 chain C region, Ig lambda-2 chain C region, angiogenin, Bad, bcl-w (Bcl2-like 2), casper, caspases 1, 3, 7, 8, 11, and 14, CRADD, cyclin-dependent kinase inhibitor p21 Waf1, DAXX (fas-binding protein), DR5 (TRAIL death-inducing receptor), Fas ligand, IAP 1 and 2 (inhibitor of apoptosis proteins 1 and 2), fibroblast growth factor, G-CSF, GADD45 (DNA-damage inducible transcript 1), HGF (hepatocyte growth factor), ILs 3, 4, 5, 6, 7, 9, 10, 12α, 15, and 18, mdm2, NFκb1, NF-κB inducing kinase, p53 responsive protein, PDGFα, retinoblastoma supsceptibility protein, RIP (cell death protein), thrombospondin 3, TNFβ, TRAF2 (TNF receptor associated factor 2), (TRAF3 (death adaptor molecule), TRAF6 (CD40 associated factor), Trail (TNF-related apoptosis inducing ligand), TRIP (TRAF-interacting protein), tumor necrosis factor I and II receptors, and VEGF-B, C, D and I, whereby said inhibiting is carried out with a polyphenol and modifies the activity of the protein, thereby treating the cancerous condition.

2. The method according to claim 1 wherein the step of inhibiting includes binding a compound to a C-terminal region of hsp90.

3. A method of treating a cancerous condition comprising:
inhibiting an interaction between hsp90 and a protein selected from the group consisting of p53, Sp1, death domain kinase RIP, insulin-like growth factor 1 receptor, mdm2, and thrombin receptor, whereby said inhibiting modifies the activity of the protein and thereby treats the cancerous condition.
